(19) United States
(12) Patent Application Publication
(10) Pub. No.: US 2007/0105870 A1
(43) Pub. Date: May 10, 2007

(54) Piperazine derivatives which exhibit activity as serotonin and noradrenaline re-uptake inhibitors

(75) Inventors: Gerwyn Bish, Sandwich (GB); Alan Daniel Brown, Sandwich (GB); Paul Vincent Fish, Sandwich (GB); Michael Jonathan Fray, Sandwich (GB); Alan Stobie, Sandwich (GB); Florian Wakenhut, Sandwich (GB); Gavin Alistair Whitlock, Sandwich (GB)

Correspondence Address: WARNER-LAMBERT COMPANY, 2800 PLYMOUTH RD, ANN ARBOR, MI, 48105, US

(73) Assignee: Pfizer Inc.

(21) Appl. No.: 10/586,029

(22) PCT Filed: Jan. 3, 2005
(86) PCT No.: PCT/IB05/00016

§ 371 Date: Jan. 17, 2007

Related U.S. Application Data
(60) Provisional application No. 60/545,554, filed on Feb. 17, 2004.

(30) Foreign Application Priority Data
Jan. 13, 2004 (GB) 0400699.5

(51) Int. Cl. A61K 31/495; C07D 241/04

(52) U.S. Cl. 514/255.04; 544/396

(57) ABSTRACT

A compound of formula (I), wherein R¹ is H; R² is aryl, het, C₃₋₈cycloalkyl, C₁₋₆alkyl, (CH₂)₂aryl or R⁴, wherein each of the cycloalkyl, aryl, het and R⁴ groups is optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkyISO₂, C₁₋₄alkyl-S-C₁₋₄alkyl, C₁₋₄alkyl-S—, C₁₋₄alkyINR¹⁰R¹¹ and NR¹⁰R¹¹; or R¹ and R², together with the carbon atom to which they are bound, form a 5- or 6-membered carbocycle or a 5- or 6-membered heterocycle containing at least one N, O or S heteroatom; R³ is aryl, het or R⁴, each optionally substituted by at least one substituent independently selected from C₁₋₆-alkyl, C₁₋₆-alkoxy, het, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkyISO₂, C₁₋₄alkyl-S-C₁₋₄alkyl, C₁₋₄alkyl-S—, C₁₋₄alkylNR¹⁰R¹¹and NR¹⁰R¹¹; R⁴ is a phenyl group fused to a 5- or 6 membered carbocycle, or a phenyl group fused to a 5- or 6-membered heterocycle containing at least one N, O or S heteroatom; R⁵ is H or C₁₋₆alkyl; R¹⁰ and R¹¹ are the same or different and are independently H or C₁₋₄alkyl; A is a C₁₋₃alkylene chain which is optionally substituted by OH, C₁₋₄alkyl or C₁₋₄alkoxy; x is an integer from 1 to 3; y is 1 or 2; z is an integer from 1 to 3; aryl is phenyl, naphthyl, anthracyl or phenanthryl; and het is an aromatic or non-aromatic 4, 5- or 6-membered heterocycle which contains at least one N, O or S heteroatom, optionally fused to a 5- or 6-membered carbocycle or a second 4, 5- or 6-membered heterocycle which contains at least one N, O or S heteroatom.

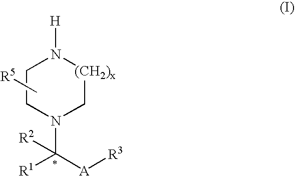

This invention relates to novel amine compounds which inhibit monoamine re-uptake, to processes for their preparation, to pharmaceutical compositions containing them and to their use in medicine.

The compounds of the invention exhibit activity as both serotonin and noradrenaline re-uptake inhibitors and therefore have utility in a variety of therapeutic areas. For example, the compounds of the invention are of use in the treatment of disorders in which the regulation of monoamine transporter function is implicated; more particularly disorders in which inhibition of re-uptake of serotonin or noradrenaline is implicated; and especially disorders in which inhibition of both serotonin and noradrenaline is implicated, such as urinary incontinence.

According to a first aspect, the invention provides a compound of Formula I, as defined below in Integer 1.
Integer 1. 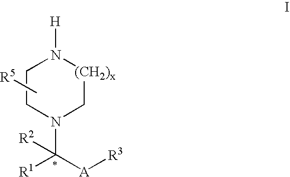
and pharmaceutically and/or veterinarily acceptable derivatives thereof, wherein:
R¹ is H;
R² is aryl, het, C₃₋₈cycloalkyl, C₁₋₆alkyl, (CH₂)₂aryl or R⁴, wherein each of the cycloalkyl, aryl, het and R⁴ groups is optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkyl-SO₂-, C₁₋₄alkyl-S-C₁₋₄alkyl, C₁₋₄alkyl-S—, C₁₋₄alkylNR¹⁰R¹¹ and NR¹⁰R¹¹;
or R¹ and R², together with the carbon atom to which they are bound, form a 5- or 6-membered carbocyclic ring or a 5- or 6-membered heterocyclic ring containing at least one N, O or S heteroatom;
where R¹ and R² are different, * represents a chiral centre;
R³ is aryl, het or R⁴, each optionally substituted by at least one substituent independently selected from C₁₋₆alkyl, C₁₋₆alkoxy, het, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂, C₁₋₄alkyl-S-C₁₋₄alkyl, C₁₋₄alkyl-S-, C₁₋₄alkylNR¹⁰R¹¹ and NR¹⁰R¹¹;
R⁴ is a phenyl group fused to a 5- or 6-membered carbocyclic group, or a phenyl group fused to a 5- or 6-membered heterocyclic group containing at least one N, O or S heteroatom;
R⁵ is H or C₁₋₆alkyl;
R¹⁰ and R¹¹ are the same or different and are independently H or C₁₋₄alkyl;
A is a C₁₋₃alkylene chain which is optionally substituted by OH, C₁₋₄alkyl or C₁₋₄alkoxy;
x is an integer from 1 to 3;
y is 1 or 2;
z is an integer from 1 to 3;
aryl is phenyl, naphthyl, anthracyl or phenanthryl; and
het is an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom, provided that when R¹ is H, R² is phenyl, A is CH₂ and x is 1, R³ is not 3-hydroxyphenyl or 3-(C₁₋₄alkoxy)phenyl.

Alternative embodiments of the invention are defined below with reference to Integers 2 to 23.

Integer 2 provides a compound according to Integer 1, wherein R¹ is H.

Integer 3 provides a compound according to Integer 1 or Integer 2, wherein R is aryl, het or C₃₋₈cycloalkyl, each optionally substituted as indicated in Integer 1.

Integer 4 provides a compound according to Integer 3, wherein R² is aryl, het or C₃₋₆cycloalkyl, each optionally substituted as indicated in Integer 1.

Integer 5 provides a compound according to Integer 4, wherein R² is aryl or het, each optionally substituted as indicated in Integer 1.

Integer 6 provides a compound according to Integer 5, wherein R² is aryl, optionally substituted as indicated in Integer 1.

Integer 7 provides a compound according to Integer 6, wherein R² is phenyl, optionally substituted as indicated in Integer 1.

Integer 8 provides a compound according to any of Integers 1 to 7, wherein R² is optionally substituted by at least one substituent independently selected from C₁₋₆alkyl, C₁₋₆alkoxy, OH, halo, CF₃, CN, when R² contains a cycloalkyl, aryl or het group.

Integer 9 provides a compound according to any of Integers 1 to 8, wherein R³ is aryl or R⁴ each optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂ and C₁₋₄alkyl-S-C₁₋₄alkyl.

Integer 10 provides a compound according to Integer 9, wherein R³ is optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy.

Integer 11 provides a compound according to Integer 10, wherein R³ is optionally substituted by at least one substituent independently selected from C₁₋₆alkyl, C₁₋₆alkoxy, OH, halo, CF₃.

Integer 12 provides a compound according to any one of Integers 9 to 11, wherein R³ is aryl, optionally substituted as indicated in any of Integers 9 to 11.

Integer 13 provides a compound according to Integer 12, wherein R³ is aryl, optionally substituted by C₁₋₃alkoxy or halo.

Integer 14 provides a compound according to Integer 12 or Integer 13, wherein R³ is phenyl, optionally substituted as indicated in any of Integers 9to 13.

Integer 15 provides a compound according to any of Integers 1 to 14, wherein R⁵ is H or C₁₋₃alkyl.

Integer 16 provides a compound according to Integer 15, wherein R⁵ is H, Me or Et.

Integer 17 provides a compound according to Integer 16, wherein R⁵ is H.

Integer 18 provides a compound according to any of Integers 1 to 17, wherein A is a C₁₋₃alkylene chain optionally substituted by OH.

Integer 19 provides a compound according to Integer 18, wherein A is a methylene (—CH₂—) group optionally substituted by OH.

Integer 20 provides a compound according to Integer 19, wherein A is an unsubstituted methylene group.

Integer 21 provides a compound according to any of Integers 1 to 20, herein x is 1.

Integer 22 provides a compound according to any of Integers 1 to 21, wherein y is 1.

Integer 23 provides a compound according to any of Integers 1 to 22, wherein z is 1.

The substituent R⁴ is defined in the above Integers as a phenyl group fused to a 5- or 6-membered carbocyclic group, or a phenyl group fused to a 5- or 6-membered heterocyclic group containing at least one N, O or S heteroatom. However, in certain embodiments, or in connection with any of the Integers mentioned above, this definition may be limited to a phenyl group fused to a 6-membered carbocyclic group, or a phenyl group fused to a 5- or 6-membered heterocyclic group containing at least one N or O heteroatom.

In any of the above Integers, the term “aryl” means phenyl, naphthyl, anthracyl or phenanthryl. However, in certain embodiments, or in connection with any of the Integers mentioned above, the definition of “aryl” may be limited to phenyl or naphthyl.

The term “het” is defined in the above Integers as an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom. However, in certain embodiments of the invention, or in connection with any of the Integers mentioned above, this may be limited to an aromatic or non-aromatic 5- or 6-membered heterocycle which contains at least one N or O heteroatom, optionally fused to a 5- or 6-membered carbocyclic group or a second 5- or 6-membered heterocycle which contains at least one N or O heteroatom; or an aromatic or non-aromatic 5- or 6-membered heterocycle which contains at least one N heteroatom, optionally fused to a 5- or 6-membered carbocyclic group or a second 5- or 6-membered heterocycle which contains at least one N heteroatom. In the preceding definitions, the second heterocycle, to which the first heterocycle may be fused, may be either aromatic or non-aromatic.

In embodiments where R¹ and R² are different, * represents a chiral centre and may be either the R or the S stereochemical configuration. Racemic mixtures of chiral compounds according to the invention may be produced and are within the scope of the invention as claimed.

A further embodiment of the invention provides a compound of Formula Ia as defined below: 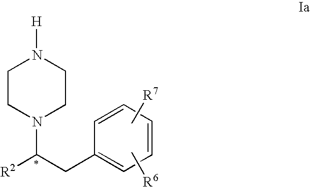
and pharmaceutically and/or veterinarily acceptable derivatives thereof, wherein:
R² is as defined above in respect of Formula I; and
R⁶ and R⁷ are the same or different and are independently selected from H, C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂, C₁₋₄alkyl-S-C₁₋₄alkyl, C₁₋₄alkyl-S—, C₁₋₄alkylNR¹⁰R¹¹ and NR¹⁰R¹¹, wherein R¹⁰ and R¹¹ are as defined above with respect to Formula I; or R⁶ and R⁷ together represent a 5- or 6-membered aromatic or non-aromatic carbocyclic ring fused to the phenyl group; or R⁶ and R⁷ together represent a 4-, 5- or 6-membered aromatic or non-aromatic heterocycle fused to the phenyl group, wherein the heterocycle contains at least one N, O or S heteroatom.

It should be noted that there may be more than one R⁶ and/or more than one R⁷ substituent. Thus, the phenyl ring may substituted by up to 4 substituents which may be the same or different, provided they are each selected from the list of possible substituent groups list above. Thus, R⁶ and R⁷ may be read as (R⁶)ₙ and (R⁷)ₘ respectively, wherein the sum of m+n is no more than 4.

In the compounds of Formula Ia, R² may be optionally substituted by at least one substituent independently selected from C₁₋₆alkyl, C₁₋₆alkoxy, OH, halo, CF₃, CN, when R² contains a cycloalkyl, aryl or het group.

Alternatively, R² may be aryl, a 5- or 6-membered aromatic or non-aromatic heterocycle containing at least one N or O heteroatom, C₁₋₆alkyl, C₃₋₆cycloalkyl or —(CH₂),aryl, wherein z is an integer from 1 to 3 and aryl is as defined above.

In certain embodiments in relation to Formula Ia, R⁶ and R⁷ may be the same or different and are independently selected from H, C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl and C₁₋₄alkoxy-C₁₋₄alkoxy; or R⁶ and R⁷ together represent a 5- or 6-membered aromatic or non-aromatic carbocyclic ring fused to the phenyl group; or R⁶ and R⁷ together represent a 5- or 6-membered aromatic or non-aromatic heterocycle fused to the phenyl group, wherein the heterocycle contains at least one N or O heteroatom.

A still further embodiment of the invention provides a compound of Formula Ib as defined below: 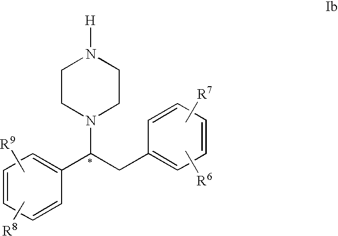
and pharmaceutically and/or veterinarily acceptable derivatives thereof, wherein:
R⁶ and R⁷ are the same or different and are independently selected from H, C₁₋₆alkyl, C₁₋₆alkoxy, OH, halo, CF₃, OCF₃, OCHF₂, O(CH₂)_{y}CF₃, CN, CONH₂; CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂ and C₁₋₄alkyl-S-C₁₋₄alkyl; or R⁶ and R⁷ together represent a 5- or 6-membered aromatic or non-aromatic carbocyclic ring fused to the phenyl group; or R⁶ and R⁷ together represent a 4-, 5- or 6-membered aromatic or non-aromatic heterocycle fused to the phenyl group, wherein the heterocycle contains at least one N, O or S heteroatom; and
R⁸ and R⁹ are the same or different and are independently selected from H, C₁₋₆alkyl, C₁₋₆alkoxy, OH, halo, CF₃, OCF₃, OCHF₂, O(CH₂)_{y}CF₃, CN; or R⁸ and R⁹ together represent a 5- or 6-membered aromatic or non-aromatic carbocyclic ring fused to the phenyl group; or R⁸ and R⁹ together represent a 4-, 5- or 6-membered aromatic or non-aromatic heterocycle fused to the phenyl group, wherein the heterocycle contains at least one N, O or S heteroatom.

It should be noted that there may be more than one R⁶ and/or more than one R⁷ substituent. Thus, the phenyl ring may substituted by up to 4 substituents which may be the same or different, provided they are each selected from the list of possible substituent groups list above. Thus, R⁶ and R⁷ may be read as (R⁶)ₙ and (R⁷)ₘ respectively, wherein the sum of m+n is no more than 4.

The same is true for R⁸ and R⁹, which also may be read as (R⁸)ₚ and (R⁹)_{q}, wherein the sum of p+q is no more than 4.

In certain embodiments in relation to the compounds of Formula Ib, R⁶ and R⁷ may be the same or different and are independently selected from H, C₁₋₆alkyl, C₁₋₆alkoxy, OH, halo, CF₃, OCF₃, OCHF₂, and O(CH₂)_{y}CF₃; or R⁶ and R⁷ together represent a 5- or 6-membered aromatic or non-aromatic carbocyclic ring fused to the phenyl group or R⁶ and R⁷ together represent a 5- or 6-membered aromatic or non-aromatic heterocycle fused to the phenyl group, wherein the heterocycle contains at least one N or O heteroatom; and

R⁸ and R⁹ are the same or different and are independently selected from H, C₁₋₆alkyl, C₁₋₆alkoxy, OH, halo, CF₃, OCF₃, OCHF₂, and O(CH₂)_{y}CF₃; or R⁸ and R⁹ together represent a 5- or 6-membered aromatic or non-aromatic carbocyclic ring fused to the phenyl group or R⁸ and R⁹ together represent a 5- or 6-membered aromatic or non-aromatic heterocycle fused to the phenyl group, wherein the heterocycle contains at least one N or O heteroatom.

In a specific embodiment, the invention provides a compound of Formula Ib, wherein R⁶ is OEt and R⁷, R⁸ and R⁹ are each H. In particular, R⁶ may be 2-ethoxy.

Example compounds within the scope of the invention are as follows:

1-{1-Phenyl-2-[2-(trifluoromethoxy)phenyl]ethyl}piperazine ditrifluoroacetate,

1-{1-Phenyl-2-[2-chloro-6-fluorophenyl]ethyl}piperazine ditrifluoroacetate,

1-{1-Phenyl-2-[2-chlorophenyl]ethyl}piperazine ditrifluoroacetate,

1-{1-(3-Fluorophenyl)-2-[2-(trifluoromethoxy)phenyl]ethyl}piperazine,

1-{2-[2-(Difluoromethoxy)phenyl]-1-phenylethyl}piperazine,

1-{1-(4-Fluorophenyl)-2-[2-(trifluoromethoxy)phenyl]ethyl}piperazine,

1-{1-(2-Fluorophenyl)-2-[2-(trifluoromethoxy)phenyl]ethyl}piperazine

1-{2-[2-(Difluoromethoxy)phenyl]-1-phenylethyl}piperazine dihydrochloride,

1-[2-(2-Chlorophenyl)-1-phenylethyl]piperazine dihydrochloride,

1-[2-(2-Methoxyphenyl)-1-phenylethyl]piperazine dihydrochloride, and

1-[2-(2-Ethoxyphenyl)-1-phenylethyl]piperazine dihydrochloride

Further embodiments of the invention include the following compounds:

1-{2-(3-methoxyphenyl)-1-[3-(trifluoromethyl)phenyl]ethyl}piperazine

1-[2-(2-ethoxyphenyl)-1-pyridin-3-ylethyl]piperazine

1-[2-(3-chlorophenyl)-1-phenylethyl]piperazine

1-[2-(2-ethoxyphenyl)-1-phenylethyl]piperazine

1-[2-(2,5-dichlorophenyl)-1-phenylethyl]piperazine

1-[2-(2,3-dichlorophenyl)-1-phenylethyl]piperazine

1-[2-(2,3-dichlorophenyl)-1-pyridin-3-ylethyl]piperazine

1-{1-phenyl-2-[2-(trifluoromethyl)phenyl]ethyl}piperazine

1-[2-(2-chlorophenyl)-1-(4-fluorophenyl)ethyl]piperazine

1-[2-(2-chlorophenyl)-1-(3-fluorophenyl)ethyl]piperazine

1-[2-(2-bromophenyl)-1-phenylethyl]piperazine

1-[2-(2-chlorophenyl)-1-(2-fluorophenyl)ethyl]piperazine

1-[2-(2,3-dichlorophenyl)-1-pyridin-4-ylethyl]piperazine

1-{1-phenyl-2-[2-(trifluoromethoxy)phenyl]ethyl}piperazine

1-[2-(2-ethoxyphenyl)-1-(3-fluorophenyl)ethyl]piperazine

1-[2-(2-ethoxyphenyl)-1-(4-fluorophenyl)ethyl]piperazine

1-[2-(2-ethoxyphenyl)-1-(2-fluorophenyl)ethyl]piperazine

1-[1-(4-fluorophenyl)-2-(2-methoxyphenyl)ethyl]piperazine

1-[1-(3-fluorophenyl)-2-(2-methoxyphenyl)ethyl]piperazine

1-[1-(2-fluorophenyl)-2-(2-methoxyphenyl)ethyl]piperazine

1-[2-(2-methylphenyl)-1-phenylethyl]piperazine

1-[1-(4-chlorophenyl)-2-(2-methoxyphenyl)ethyl]piperazine

1-[1-(3-chlorophenyl)-2-(2-methoxyphenyl)ethyl]piperazine

1-[1-phenyl-2-(2-propoxyphenyl)ethyl]piperazine

1-{2-[2-(2-methoxyethoxy)phenyl]-1-phenylethyl}piperazine

1-(1-benzyl-2-phenylethyl)piperazine

1-{2-[2-(methoxymethyl)phenyl]-1-phenylethyl}piperazine

1-[2-(2-ethylphenyl)-1-phenylethyl]piperazine

1-{1-phenyl-2-[2-(2,2,2-trifluoroethoxy)phenyl]ethyl}piperazine

1-[2-(2-fluoro-6-methoxyphenyl)-1-phenylethyl]piperazine

1-{2-[2-(difluoromethoxy)-6-fluorophenyl]-1-phenylethyl}piperazine

1-{2-[2-fluoro-6-(trifluoromethyl)phenyl]-1-phenylethyl}piperazine

1-{1-(3-fluorophenyl)-2-[2-(trifluoromethyl)phenyl]ethyl}piperazine

1-[2-(2-isopropoxyphenyl)-1-phenylethyl]piperazine

1-{1-(4-chlorophenyl)-2-[2-(difluoromethoxy)phenyl]ethyl}piperazine

(1S,2S)-1-(2-methoxyphenyl)-2-phenyl-2-piperazin-1-ylethanol

1-{1-(3-chlorophenyl)-2-[2-(difluoromethoxy)phenyl]ethyl}piperazine

1-{1-(2-chlorophenyl)-2-[2-(difluoromethoxy)phenyl]ethyl}piperazine

1-{1-(4-fluorophenyl)-2-[2-(trifluoromethyl)phenyl]ethyl}piperazine

1-{2-[2-(cyclopropyloxy)phenyl]-1-phenylethyl}piperazine

(1S,2S)-1-(2,3-dichlorophenyl)-2-phenyl-2-piperazin-1-ylethanol

(1S,2S)-1-(2-chlorophenyl)-2-phenyl-2-piperazin-1-ylethanol

(1S,2S)-1-(2-ethoxyphenyl)-2-phenyl-2-piperazin-1-ylethanol

1-[2-(2-chlorophenyl)-1-phenylethyl]-1,4-diazepane

1-(1,3-diphenylpropyl)piperazine.

According to a further aspect of the invention, there is provided one or more metabolites of the compounds of Formula I, Ia or Ib when formed in vivo.

In particular, it is believed that the compounds of Formula I, may metabolise to a compound of Formula II, wherein R1, R2, R3, R5 and A are all as defined with respect to Formula I above:

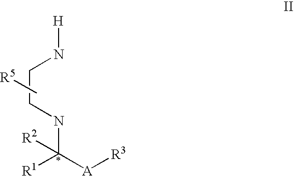

The metabolites of Formula II are also considered to constitute an aspect of the present invention.

By pharmaceutically and/or veterinarily acceptable derivative it is meant any pharmaceutically or veterinarily acceptable salt, solvate, prodrug (e.g. ester or amide), or salt or solvate of such prodrug (e.g. a salt or solvate of an ester or amide), of the compounds of Formula I, Ia or Ib or any other compound which upon administration to the recipient is capable of providing (directly or indirectly) a compound of Formula I, Ia or Ib

For pharmaceutical or veterinary use, the salts referred to above will be the pharmaceutically or veterinarily acceptable salts, but other salts may find use, for example in the preparation of compounds of Formula I, Ia or Ib and the pharmaceutically or veterinarily acceptable salts thereof.

The aforementioned pharmaceutically or veterinarily acceptable salts include the acid addition and base salts thereof.

Suitable acid addition salts are formed from acids which form non-toxic salts. Examples include the acetate, aspartate, benzoate, besylate, bicarbonate/carbonate, bisulphate/sulphate, camsylate, citrate, edisylate, hemiedisylate, esylate, fumarate, gluceptate, gluconate, glucuronate, hibenzate, hydrochloride/chloride, hydrobromide/bromide, hydroiodide/iodide, isethionate, lactate, malate, maleate, malonate, mesylate, methylsulphate, 2-napsylate, nicotinate, nitrate, orotate, pamoate, phosphate/hydrogen phosphate/dihydrogen phosphate, saccharate, stearate, succinate, tartrate and tosylate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of Formula I, Ia, or Ib may be readily prepared by mixing together solutions of the compound and the desired acid or base, as appropriate. The salt may precipitate from solution and be collected by filtration or may be recovered by evaporation of the solvent. The degree of ionisation in the salt may vary from completely ionised to almost non-ionised.

Pharmaceutically acceptable solvates in accordance with the invention include hydrates and solvates of the compounds of Formula I, Ia, or Ib.

Also within the scope of the invention are complexes such as clathrates, drug-host inclusion complexes wherein, in contrast to the aforementioned solvates, the drug and host are present in stoichiometric or non-stoichiometric amounts. Also included in this invention are complexes of the pharmaceutical drug which contain two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of Formula I, Ia, or Ib may be modified to provide pharmaceutically or veterinarily acceptable derivatives thereof at any of the functional groups in the compounds. Examples of such derivatives are described in: Drugs of Today, Volume 19, Number 9, 1983, pp 499-538; Topics in Chemistry, Chapter 31, pp 306-316; and in “Design of Prodrugs” by H. Bundgaard, Elsevier, 1985, Chapter 1 (the disclosures in which documents are incorporated herein by reference) and include: esters, carbonate esters, hemi-esters, phosphate esters, nitro esters, sulfate esters, sulfoxides, amides, sulphonamides, carbamates, azo-compounds, phosphamides, glycosides, ethers, acetals and ketals.

It will be further appreciated by those skilled in the art, that certain moieties, known in the art as “pro-moieties”, for example as described by H. Bundgaard in “Design of Prodrugs” (ibid) may be placed on appropriate functionalities when such functionalities are present within compounds of the invention.

The compounds of Formula I, Ia or Ib may contain one or more chiral centres, for example by virtue of the asymmetric carbon atom defined by certain meanings of R¹ and R². Such compounds exist in a number of stereoisomeric forms (e.g. in the form of a pair of optical isomers, or enantiomers). It is to be understood that the present invention encompasses all isomers of the compounds of the invention, including all geometric, tautomeric and optical forms, and mixtures thereof (e.g. tautomeric or racemic mixtures).

The compounds of the invention may exist in one or more tautomeric forms. All tautomers and mixtures thereof are included in the scope of the present invention. For example, a claim to 2-hydroxypyridinyl would also cover its tautomeric form α-pyridonyl.

It is to be understood that the present invention includes radiolabelled compounds of Formula I, Ia or Ib

The compounds of Formula I, Ia or Ib and their pharmaceutically and veterinarily acceptable derivatives thereof may also be able to exist in more than one crystal form, a characteristic known as polymorphism. All such polymorphic forms (“polymorphs”) are encompassed within the scope of the invention. Polymorphism generally can occur as a response to changes in temperature or pressure or both, and can also result from variations in the crystallisation process. Polymorphs can be distinguished by various physical characteristics, and typically the x-ray diffraction patterns, solubility behaviour, and melting point of the compound are used to distinguish polymorphs.

Unless otherwise indicated, any alkyl group may be straight or branched and is of 1 to 8 carbon atoms, such as 1 to 6 carbon atoms or 1 to 4 carbon atoms, for example a methyl, ethyl, n-propyl, i-propyl, n-butyl, i-butyl, s-butyl or t-butyl group. Where the alkyl group contains more than one carbon atom, it may be unsaturated. Thus, the term C₁₋₆ alkyl includes C₂₋₆ alkenyl and C₂₋₆ alkynyl. Similarly, the term C₁₋₈alkyl includes C₂₋₈alkenyl and C₂₋₈alkynyl, and the term C₁₋₄alkyl includes C₂₋₄alkenyl and C₂₋₄alkynyl.

The term halogen is used to represent fluorine, chlorine, bromine or iodine.

Unless otherwise indicated, the term het includes any aromatic, saturated or unsaturated 4-, 5- or 6-membered heterocycle which contains up to 4 heteroatoms selected from N, O and S. Examples of such heterocyclic groups included furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazapinyl, thiazepinyl, diazepinyl and thiazolinyl. In addition, the term heterocycle includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazdinyl, benzothiazolyl, phthalimido, benzodiazepinyl, indolyl and isoindolyl. The terms het, heterocyclyl and heterocyclic should be similarly construed.

For the avoidance of doubt, unless otherwise indicated, the term “substituted” means substituted by one or more defined groups. In the case where groups may be selected from a number of alternative groups, the selected groups may be the same or different. Further, the term “independently” means that where more than one substituent is selected from a number of possible substituents, those substituents may be the same or different.

Hereinafter, the compounds of Formula I, Ia and Ib, and their pharmaceutically and veterinarily acceptable derivatives, the radiolabelled analogues of the foregoing, the isomers of the foregoing, and the polymorphs of the foregoing, are referred to as “compounds of the invention”.

In one embodiment of the invention, “compounds of the invention” are the pharmaceutically and veterinarily acceptable derivatives of compounds of Formula I, Ia or Ib, such as the pharmaceutically or veterinarily acceptable salts or solvates of compounds of Formula I, Ia or Ib, (e.g. pharmaceutically or veterinarily acceptable salts of compounds of Formula I, Ia or Ib).

In a still further embodiment of the invention, there is provided a compound of Formula I, Ia or Ib which is an inhibitor of serotonin and/or noradrenaline monoamine re-uptake, having SRI or NRI Ki values of 200 nM or less. In a further embodiment, the compound has SRI and/or NRI Ki values of 100 nM or less. In a yet further embodiment, the compound has SRI or NRI Ki values of 50 nM or less. In a still further embodiment, the compound has SRI and NRI Ki values of 50 nM or less. In a still yet further embodiment, the compound has SRI and NRI Ki values of 25 nM or less.

According to Scheme 1, compounds of Formula I may be prepared by analogy with the methods of Nishimura et.al. DE 2610433 or Natsuka et.al. J. Med. Chem. 1987, 30, 10, 1779-1787.

Alternatively the compounds of Formula I may be prepared according to the methods of scheme 1 shown below, when R¹ represents H and A is unsubstituted, and R², R³ and x are as previously defined.

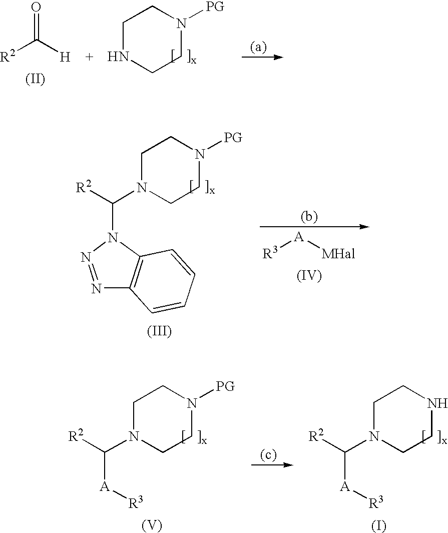

PG represents a suitable nitrogen protecting group, typically Boc, benzyl or CBz, and preferably Boc. M represents a suitable reactive metal, such as Zn or Mg, and Hal represents a halogen, typically Br or Cl and preferably Cl.

The compounds of formula (II) and (IV) are either available commercially, or may be prepared from commercial materials using standard chemical transformations.

Step (a)—Mannich Reaction

Preparation of the compound of formula (III) may be achieved by reaction of benzotriazole, a suitable protected cyclic amine, and an aldehyde (R²CHO) in equimolar amounts in a suitable solvent, such as benzene, THF or toluene, at elevated temperature and with concomitant removal of water (eg using a suitable drying agent, or under Dean and Stark conditions).

Preferred conditions are: 1 eq benzotriazole, 1 eq protected cyclic amine, 1 eq aldehyde in toluene at reflux under Dean and Stark conditions for about 5 hours.

Step (b)—

Compounds of formula (V) may be prepared by reaction of the benzotriazole adduct of formula (III) with a suitable organometallic reagent (R³MHal), in a suitable solvent such as toluene or THF, by analogy with the method of Katritzky et.al. (Tetrahedron, 1991, 47, 2683 or Chem. Soc. Rev. 363 (1994) and references therein).

Preferred conditions when M represents Zn are:

2eq R³-A-ZnCl (IV) (optionally generated in-situ), in THF and toluene at rt for 2-18 hrs.

When M represents Mg:

2.0-2.1 eq R³-A-MgCl (optionally generated in situ) in THF, optionally with toluene as a co-solvent, at between −70° C. and 0° C. for about 2 hrs.

Optionally steps (a) and (b) may be performed in a “one-pot” reaction.

Step (c)—Deprotection of N Protectinq Group

Deprotection of compound (V) to provide the compound of formula (I) is undertaken using standard methodology, as described in “Protecting Groups in Organic Synthesis” by T. W. Greene and P. Wutz. PG is preferably Boc. The typical conditions for deprotection are treatment with a strong acid (eg HCl or TFA) in a suitable solvent, such as DCM, dioxan or ether at between 0° C. and rt. The preferred conditions are: TFA:DCM (1:10 to 1:1 by volume) at between 0° C. and rt for up to 18 hrs, Or, aq. HCl in toluene or THF at between 0° C. and rt for up to 48 hrs, Or, 4M HCl in dioxan and DCM at rt for 18 hrs.

Compounds of formula (I), where A represents as C₁-C₃ alkylene chain substituted by OH and R², R³ and x are as previously defined, may be prepared according to scheme 2. 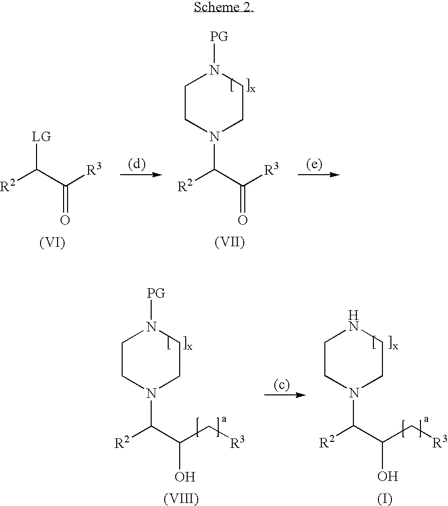
LG represents a suitable leaving group, such as halo or mesylate, typically bromo or chloro and preferably bromo.

a represents 0, 1 or 2.

Compounds of formula (VI) are either available commercially or may be prepared by analogy with the method of Shimokawa et.al. (J. Med. Chem. 1979, 22, 1, 58-63).

Step (d)-Amination

Compounds of formula (VII) may be prepared from compounds of formula (VI) by reaction with an excess of suitable protected cyclic amine, in the presence of a base (eg K₂CO₃, or 3° amine base such as Et₃N, NMM, Hünig's base) in a suitable solvent, such as THF, MeCN, DMF or EtOH at between rt and about 70° C., for up to 72 hours.

Preferred conditions are:

1-1.1eq Boc-piperazine, 3eq Et₃N, in EtOH at 60° C. for about 3 hrs, or 1 eq Boc-piperazine, 1.5-3eq K₂CO₃ in THF or DMF at rt for 18-72 hrs.

Step (e)—Reduction

Compounds of formula (VII) may be reduced, using a suitable reducing agent such as NaBH₄ or LiAlH₄ in a suitable solvent at rt to provide the alcohol of formula (VII).

Preferred conditions are:

2eq NaBH₄, in MeOH at rt for 18 hrs.

Compounds of formula (I) may be obtained by deprotection of the N protecting group of the compounds of formula (VIII), using the methods of step (c), as described previously in scheme 1.

Compounds of formula (VII) may alternatively be prepared, where A represents a C₁-C₃ alkylene chain substituted by OH, as described in scheme 3.

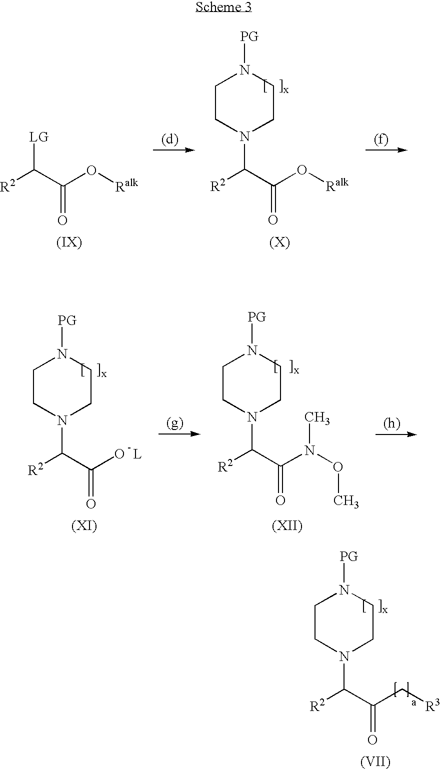

R^{alk} represents a C₁₋₆ alkyl or benzyl group, typically a C₁₋₄ group and preferably Me. L represents an alkali metal, preferably Na. a represents 0, 1 or 2

LG is a suitable leaving group, such as halo or mesylate, preferably Br. Compounds of formula (IX) are commercially available.

Compounds of formula (X) may be prepared from the compounds of formula (IX) by reaction with a suitable protected cyclic amine, preferably Boc-piperazine, according to the method of step (d) as previously described in scheme 2.

Step (f)—Carboxylate Formation

Compounds of formula (X) may be treated with a suitable strong base, such as an alkali metal hydroxide (eg NaOH, LiOH, KOH) in aqueous solvent to provide the compounds of formula (XI).

Preferred conditions are:

1 eq NaOH, H₂O: MeOH (1:1 by volume) at rt for 18 hrs.

Step (g)—Weinreb Amide Formation

Reaction of the compounds of formula (XI) with CH₃NHOCH₃ in the presence of a conventional coupling agent (e.g. WSCDI, DCC), optionally in the presence of HOBT or HOAT, with an excess of acid acceptor (e.g. Et₃N, Hünig's base) in a suitable solvent (e.g. EtOAc, THF, DCM) at rt. provides the compounds of formula (XII)

Preferred conditions are:

1.1 eq CH₃NHOCH₃, 1.2eq WSCDI, 1.5eq HOBT, 3.5 eq Et₃N in DCM for 18 hrs at rt.

Step (h)—Formation of Ketone

Compounds of formula (VII) may be prepared by reaction of the compounds of formula (XII) with a suitable organometallic reagent (typically BuLi) followed by treatment with R³Hal, (Hal is typically Br or I and preferably I)

Preferred conditions are: 2.05eq n-BuLi, 2 eq R³I in THF at between −78° C. and rt for about 2 hrs.

Compounds of formula (VIII) may alternatively be prepared according to the methods described in scheme 4.

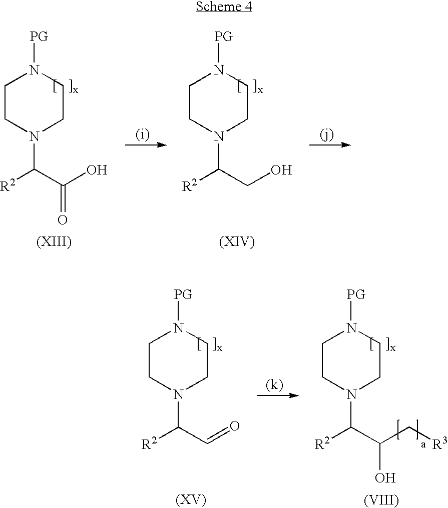

a represents 0,1 or 2.

Compounds of formula (XIII) may be obtained by treatment of the compounds of formula (XI) with aqueous acid under standard conditions.

Step (i)—Reduction of Carboxylic Acid

Compounds of formula (XIV) may be prepared by reduction of the compounds of formula (XIII) using a suitable reducing agent, such as a metal hydride or borane reducing agent (e.g. DIBAL, LiAIH₄, BH₃) in a suitable solvent (e.g. THF, toluene) at between −78° C. and rt or by hydrogenation with a copper chromite catalyst in a suitable solvent at high temperature and pressure.

Preferred conditions are:

2eq BH₃ in THF at between 0° C. and rt for upto 18 hrs.

Step (j)—Oxidation of Alcohol

Oxidation of the alcohol of formula (XIV) may be achieved using a suitable mild oxidising agent such as Dess-Martin periodinane as described in J. Am. Chem. Soc. 113, 7277, 1991, or tetra-n-propylammonium perruthenate(VII)/NMO as described in Synthesis 639, 1994 or under Swern conditions as described in Org. Synth. Coll. 7, 258, 1990 to provide the aldehyde of formula (XV).

Preferred conditions are:

1.5 eq (COCl)₂, 2.5 eq DMSO, 5eq Hünig's base, in DCM between −78° C. and rt.

Step) (k)—Grignard Reaction

Reaction of the compounds of formula (XV) with a suitable Grignard reagent (R³MgHal, Hal represents Cl or Br), optionally generated in-situ, in a suitable solvent such as THF or ether may provide the compounds of formula (VIII).

Preferred conditions are:

1.2-2.2 eq R³MgBr, in THF at between 0° C. and rt for 18 hrs.

Compounds of formula (I), where A is substituted by OH and R², R³ and a are as previously defined may also be prepared from 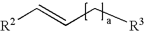
by analogy with the methods of Bolli and Ley (J. Chem. Soc. Perkin Trans. 1,1998, 2243-46).

It will be appreciated by those skilled in the art, that compounds of formula (I) where A is substituted by C₁-C₄ alkoxy, may be obtained from compounds of formula (I) (or (VIII) when a protecting group strategy is required), where A is substituted by OH using standard conditions of alkylation. For example, treatment of compound (VIII) with a suitable base, such as NaH, followed by treatment with a suitable alkylating agent, C₁-C₄Hal.

Compounds of formula (I) where R¹ and R² together with the carbon atom to which they are bound, form a 5- or 6- membered ring may be prepared according to the methods described in scheme 5.

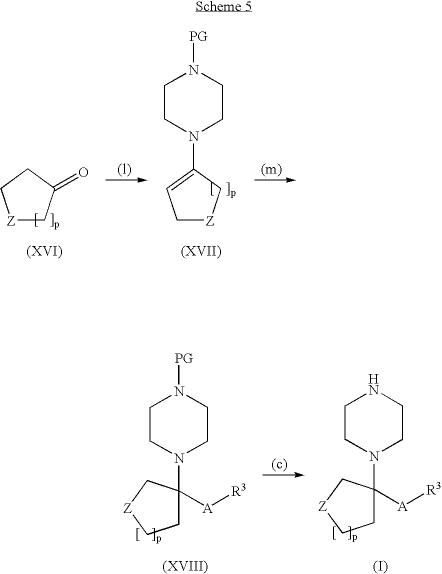

p represents 1 or 2.

Z represents N, O or S.

Compounds of formula (XVI) are available commercially.

Step (l)—Enamine Formation

The compound of formula (XVII) may be prepared by reaction of the ketone (XVI) with a protected cyclic amine by analogy with the method of Yamamoto (J. Org. Chem. 1998, 63, 377-378).

Preferred conditions are:

1.2 eq protected cyclic amine, cat. Mel, 1-1.5eq BSA in hexane at between 50-75° C. for about 4 hrs.

Step (m)—Amine Formation

Compounds of formula (XVIII) may be prepared from the compounds of formula (XVII) by treatment with benzotriazole, followed by reaction with a suitable R³-A-MHal, (M is typically Zn or Mg, and Hal is typically Cl or Br), by analogy with the method of Katritzky et. al. Synthesis, 1992, 1295.

Preferred conditions are:

1.34 eq benzotriazole in THF for 15 min-1 hr, followed by 2 eq R³-A-ZnCl for 18 hrs at rt.

Treatment of the compound of formula (XVIII) as previously described in step (c), provides the compound of formula (I).

Unless otherwise provided herein:

CDl means N,N′-carbonyidiimidazole;

WSCDI means 1-(3-dimethylaminopropyl)-3-ethylcarbodiimide hydrochloride;

DCC means N,N′-dicyclohexylcarbodiimide;

HOAT means 1-hydroxy-7-azabenzotriazole;

HOBT means 1-hydroxybenzotriazole hydrate;

Hünig's base means N-ethyidiisopropylamine;

Et₃N means triethylamine;

NMM means N-methylmorpholine;

DIBAL means diisobutylammonium hydride;

Dess-Martin periodinane means 1,1,1-triacetoxy-1,1-dihydro-1,2-benziodoxol-3(1 H)-one;

BSA means N,O-Bis(trimethylsilyl)acetamide;

Boc means tert-butoxycarbonyl;

CBz means benzyloxycarbonyl;

MeOH means methanol;

EtOH means ethanol;

EtOAc means ethyl acetate;

THF means tetrahydrofuran;

DMSO means dimethyl sulphoxide;

DCM means dichloromethane;

DMF means N,N-dimethylformamide;

AcOH means acetic acid; and

TFA means trifluoroacetic acid.

Certain intermediates described above are novel compounds and it is to be understood that all novel intermediates herein for further aspects of the present invention.

Racemic compounds may be separated either using preparative HPLC and a column with a chiral stationary phase, or resolved to yield individual enantiomers utilizing methods known to those skilled in the art. In addition, chiral intermediate compounds may be resolved and used to prepare chiral compounds of the invention.

The compounds of the invention are useful because they have pharmacological activity in mammals, including humans. Thus, they are useful in the treatment or prevention of disorders in which the regulation of monoamine transporter function is implicated, more particularly disorders in which inhibition of re-uptake of serotonin or noradrenaline is implicated, and especially those in which inhibition of serotonin and noradrenaline re-uptake is implicated.

Accordingly the compounds of the invention are useful in the treatment of urinary incontinence, such as genuine stress incontinence (GSI), stress urinary incontinence (SUI) or urinary incontinence in the elderly; overactive bladder (OAB), including idiopathic detrusor instability, detrusor overactivity secondary to neurological diseases (e.g. Parkinson's disease, multiple sclerosis, spinal cord injury and stroke) and detrusor overactivity secondary to bladder outflow obstruction (e.g. benign prostatic hyperplasia (BPH), urethral stricture or stenosis); nocturnal eneuresis; urinary incontinence due to a combination of the above conditions (e.g. genuine stress incontinence associated with overactive bladder); and urinary symptoms, such as frequency and urgency.

In view of their aforementioned pharmacological activity the compounds of the invention are also us,eful in the treatment of depression, such as major depression, recurrent depression, single episode depression, subsyndromal symptomatic depression, depression in cancer patients, depression in Parkinson's patients, postmyocardial infarction depression, paediatric depression, child abuse induced depression, depression in infertile women, post partum depression, premenstrual dysphoria and grumpy old man syndrome.

In view of their aforementioned pharmacological activity the compounds of the invention are also useful in the treatment of cognitive disorders such as dementia, particularly degenerative dementia (including senile dementia, Alzheimer's disease, Pick's disease, Huntingdon's chorea, Parkinson's disease and Creutzfeldt-Jakob disease) and vascular dementia (including multi-infarct dementia), as well as dementia associated with intracranial space occupying lesions, trauma, infections and related conditions (including HIV infection), metabolism, toxins, anoxia and vitamin deficiency; mild cognitive impairment associated with ageing, particularly age associated memory impairment (AAMI), amnestic disorder and age-related cognitive decline (ARCD); psychotic disorders, such as schizophrenia and mania; anxiety disorders, such as generalised anxiety disorder, phobias (e.g. agoraphobia, social phobia and simple phobias), panic disorder, obsessive compulsive disorder, post traumatic stress disorder, mixed anxiety and depression; personality disorders such as avoidant personality disorder and attention deficit hyperactivity disorder (ADHD); sexual dysfunction, such as premature ejaculation, male erectile dysfunction (MED) and female sexual dysfunction (FSD) (e.g. female sexual arousal disorder (FSAD)); premenstrual syndrome; seasonal affective disorder (SAD); eating disorders, such as anorexia nervosa and bulimia nervosa; obesity; appetite suppression; chemical dependencies resulting from addiction to drugs or substances of abuse, such as addictions to nicotine, alcohol, cocaine, heroin, phenobarbital and benzodiazepines; withdrawal syndromes, such as those that may arise from the aforementioed chemical dependencies; cephalic pain, such as migraine, cluster headache, chronic paroxysmal hemicrania, headache associated with vascular disorders, headache associated with chemical dependencies or withdrawal syndromes resulting from chemical dependencies, and tension headache; pain; Parkinson's diseases, such as dementia in Parkinson's disease, neuroleptic-induced Parkinsonism and tardive dyskinesias); endocrine disorders, such as hyperprolactinaemia; vasospasm, such as in the cerebral vasculature; cerebellar ataxia; Tourette's syndrome; trichotillomania; kleptomania; emotional lability; pathological crying; sleeping disorder (cataplexy); and shock.

In view of their aforementioned pharmacological activity the compounds of the invention are also useful in the treatment of a number of other conditions or disorders, including hypotension; gastrointestinal tract disorders (involving changes in motility and secretion) such as irritable bowel syndrome (IBS), ileus (e.g. post-operative ileus and ileus during sepsis), gastroparesis (e.g. diabetic gastroparesis), peptic ulcer, gastroesophageal reflux disease (GORD, or its synonym GERD), flatulence and other functional bowel disorders, such as dyspepsia (e.g. non-ulcerative dyspepsia (NUD)) and non-cardiac chest pain (NCCP); and fibromyalgia syndrome.

In view of their aforementioned pharmacological activity, the compounds of the invention may also be useful in the treatment of pain. For example, pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Further examples include tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertebral discs or abnormalities of the lumber facet joints, sacroiliac joints, paraspinal muscles or the posterior longitudinal ligament

In addition, the compounds of the invention may be useful in the treatment of neuropathic pain. This is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, diabetic neuropathy, post herpetic neuralgia, back pain, cancer neuropathy, chemotherapy-induced neuropathy, HIV neuropathy, Phantom limb pain, Carpal Tunnel Syndrome, chronic alcoholism, hypothyroidism, trigeminal neuralgia, uremia, trauma-induced neuropathy, or vitamin deficiencies

Other types of pain include but are not limited to:
Inflammatory pain, such as arthritic pain, including rheumatoid arthritis (RA) and ostoearthritis (OA), and inflammatory bowel disease (IBD);
Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis;
Central pain or ‘thalamic pain’ as defined by pain caused by lesion or dysfunction of the nervous system including but not limited to central post-stroke pain, multiple sclerosis, spinal cord injury, Parkinson's disease and epilepsy;
Heart and vascular pain including but not limited to angina, myocardical infarction, mitral stenosis, pericarditis, Raynaud's phenomenon, sclerodoma, skeletal muscle ischemia;
Visceral pain, and gastrointestinal disorders, including the pain associated with dysmenorrhea, pelvic pain, cystitis and pancreatitis;
Head pain including but not limited to migraine, migraine with aura, migraine without aura, cluster headache, tension-type headache; and
Orofacial pain including but not limited to dental pain, temporomandibular myofascial pain.

Disorders of particular interest include urinary incontinence, such as mixed incontinence, GSI and USI; pain; depression; anxiety disorders, such as obsessive-compulsive disorder and post traumatic stress disorder; personality disorders, such as ADHD; sexual dysfunction; and chemical dependencies and withdrawal syndromes resulting from chemical dependencies.

Thus, according to further aspects, the invention provides:

i) a compound of the invention for use in human or veterinary medicine;

ii) a compound of the invention for use in the treatment of a disorder in which the regulation of inonoamine transporter function is implicated, such as urinary incontinence;

iii) the use of a compound of the invention in the manufacture of a medicament for the treatment of a disorder in which the regulation of monoamine transporter function is implicated;

iv) a compound of the invention for use in the treatment of a disorder in which the regulation of serotonin or noradrenaline is implicated;

v) the use of a compound of the invention in the manufacture of a medicament for the treatment of a disorder in which the regulation of serotonin and noradrenaline is implicated;

vi) a compound of the invention for use in the treatment of urinary incontinence, such as GSI or SUI;

vii) the use of a compound of the invention in the manufacture of a medicament for the treatment of urinary incontinence, such as GSI or SUI;

viii) a compound of the invention for use in the treatment of depression;

ix) the use of a compound of the invention in the manufacture of a medicament for the treatment of depression;

x) a method of treatment of a disorder in which the regulation of monoamine transporter function is implicated which comprises administering a therapeutically effective amount of a compound of the invention to a patient in need of such treatment;

xi) a method of treatment of a disorder in which the regulation of serotonin or noradrenaline is implicated which comprises administering a therapeutically effective amount of a compound of the invention to a patient in need of such treatment;

xii) a method of treatment of a disorder in which the regulation of serotonin and noradrenaline is implicated which comprises administering a therapeutically effective amount of a compound of the invention to a patient in need of such treatment;

xiii) a method of treatment of urinary incontinence, such as GSI or SUI, which comprises administering a therapeutically effective amount of a compound of the invention to a patient in need of such treatment; and

xiv) a method of treatment of depression, which comprises administering a therapeutically effective amount of a compound of the invention to a patient in need of such treatment.

It is to be appreciated that all references herein to treatment include curative, palliative and prophylactic treatment, unless explicitly stated otherwise.

The compounds of the invention may be administered alone or as part of a combination therapy. If a combination of therapeutic agents is administered, then the active ingredients may be administered either sequentially or simultaneously in separate or combined pharmaceutical formulations.

Examples of suitable agents for adjunctive therapy include:

an estrogen agonist or selective estrogen receptor modulator (e.g. HRT therapies or lasofoxifene);

an alpha-adrenergic receptor agonist, such as phenylpropanolamine or R-450;

an alpha-adrenergic receptor antagonist (e.g. phentolamine, doxazasin, tamsulosin, terazasin and prazasin), including a selective alpha_{1L}-adrenergic receptor antagonist (e.g. Example 19 of WO98/30560);

a beta-adrenergic agonist (e.g. clenbuterol);

a muscarinic receptor antagonist (e.g. tolterodine or oxybutinin), including a muscarinic M3 receptor antagonist (e.g. darifenacin);

a Cox inhibitor, such as a Cox-2 inhibitor (e.g. celecoxib, rofecoxib, valdecoxib parecoxib or etoricoxib);

a tachykinin receptor antagonist, such as a neurokinin antagonist (e.g. an NK1, NK2 or NK3 antagonist);

a beta 3 receptor agonist;

a 5HT₁ ligand (e.g buspirone);

a 5HT₁ agonist, such as a triptan (e.g. sumatriptan or naratriptan);

a dopamine receptor agonist (e.g. apomorphine, teachings on the use of which as a pharmaceutical may be found in U.S. Pat. No. 5,945,117), including a dopamine D2 receptor agonist (e.g. premiprixal, Pharmacia Upjohn compound number PNU95666; or ropinirole);

a melanocortin receptor agonist (e.g. melanotan II);

a PGE receptor antagonist;

a PGE1 agonist (e.g. alprostadil);

a further monoamine transport inhibitor, such as an noradrenaline re-uptake inhibitor (e.g. reboxetine), a serotonin re-uptake inhibitor (e.g. sertraline, fluoxtine, or paroxetine), or a dopamine re-uptake Inhibitors;

a 5-HT3 receptor antagonist (e.g. ondansetron, granisetron, tropisetron, azasetron, dolasetron or alosetron);

a phosphodiesterase (PDE) inhibitor, such as PDE2 inhibitor, (e.g. erythro-9-(2-hydroxyl-3-nonyl)-adenine or Example 100 of EP 0771799, incorporated herein by reference) and in particular a PDE5 inhibitor (e.g. sildenafil; 1-{[3-(3,4-dihydro-5-methyl-4-oxo-7-propylimidazo[5, 1-f]-as-trazin-2-yl)-4-ethoxyphenyl]sulfonyl}-4-ethylpiperazine, i.e. vardenafil, also known as Bayer BA 38-9456; or Icos Lilly's IC351, see structure below).

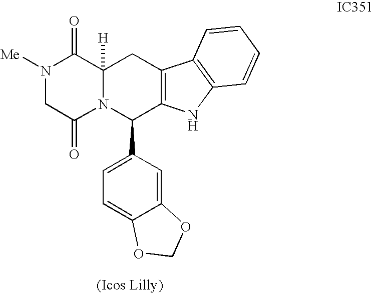

The invention thus provides, in a further aspect, a combination comprising a compound of the invention together with a further therapeutic agent.

For human use the compounds of the invention can be administered alone, but in human therapy will generally be administered in admixture with a suitable pharmaceutical excipient, diluent or carrier selected with regard to the intended route of administration and standard pharmaceutical practice.

For example, the compounds of the invention, can be administered orally, buccally or sublingually in the form of tablets, capsules (including soft gel capsules), ovules, elixirs, solutions or suspensions, which may contain flavouring or colouring agents, for immediate-, delayed-, modified-, sustained-, dual-, controlled-release or pulsatile delivery applications. The compounds of the invention may also be administered via intracavernosal injection. The compounds of the invention may also be administered via fast dispersing or fast dissolving dosage forms.

Such tablets may contain excipients such as microcrystalline cellulose, lactose, sodium citrate, calcium carbonate, dibasic calcium phosphate, glycine, and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenrate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention, and their pharmaceutically acceptable salts, may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intravenously, intra-arterially, intraperitoneally, intrathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

For oral and parenteral administration to human patients, the daily dosage level of the compounds of the invention or salts or solvates thereof will usually be from 10 to 500 mg (in single or divided doses).

Thus, for example, tablets or capsules of the compounds of the invention or salts or solvates thereof may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including PE), compounds of the invention may be taken as a single dose on an “as required” basis (i.e. as needed or desired).

Example Tablet Formulation

In general a tablet formulation could typically contain between about 0.01 mg and 500 mg of a compound according to the present invention (or a salt thereof) whilst tablet fill weights may range from 50 mg to 1000 mg. An example formulation for a 10 mg tablet is illustrated:

| Ingredient | % w/w |
|---|---|
| Free base or salt of compound | 10.000* |
| Lactose | 64.125 |
| Starch | 21.375 |
| Croscarmellose Sodium | 3.000 |
| Magnesium Stearate | 1.500 |

*This quantity is typically adjusted in accordance with drug activity and is based on the weight of the free base.

The compounds of the invention can also be administered intranasally or by inhalation and are conveniently delivered in the form of a dry powder inhaler or an aerosol spray presentation from a pressurised container, pump, spray or nebulizer with the use of a suitable propellant, e.g. dichlorodifluoromethane, trichlorofluoromethane, dichlorotetra- fluoro-ethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compounds of the invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch. They may also be administered by the ocular, pulmonary or rectal routes.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively,. they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters, wax, cetearyl alcohol, 2-octyidodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

For oral or parenteral administration to human patients the daily dosage levels of compounds of formula (I), and their pharmaceutically acceptable salts, will be from 0.01 to 30 mg/kg (in single or divided doses) and preferably will be in the range 0.01 to 5 mg/kg. Thus tablets will contain 1 mg to 0.4 g of compound for administration singly or two or more at a time, as appropriate. The physician will in any event determine the actual dosage which will be most suitable for any particular patient and it will vary with the age, weight and response of the particular patient. The above dosages are, of course only exemplary of the average case and there may be instances where higher or lower doses are merited, and such are within the scope of the invention.

Oral administration is preferred.

For veterinary use, a compound of the invention is administered as a suitably acceptable formulation in accordance with normal veterinary practice and the veterinary surgeon will determine the dosing regimen and route of administration which will be most appropriate for a particular animal.

Thus according to a further aspect, the invention provides a pharmaceutical formulation containing a compound of the invention and a pharmaceutically acceptable adjuvant, diluent or carrier.

The combinations referred to above may also conveniently be presented for use in the form of a pharmaceutical formulation and thus pharmaceutical formulations comprising a combination as defined above together with a pharmaceutically acceptable adjuvant, diluent or carrier comprise a further aspect of the invention. The individual components of such combinations may be administered either sequentially or simultaneously in separate or combined pharmaceutical formulations.

When a compound of the invention is used in combination with a second therapeutic the dose of each compound may differ from that when the compound is used alone. Appropriate doses will be readily appreciated by those skilled in the art.

The invention is illustrated by the following non-limiting examples in which the following abbreviations and definitions may be used:

| | |
|---|---|
| APCI | Atmospheric pressure chemical ionisation |
| Arbacel ® | filter agent |
| br | Broad |
| BOC | tert-butoxycarbonyl |
| CDI | carbonyldiimidazole |
| δ | chemical shift |
| d | doublet |
| Δ | heat |
| DCCI | dicyclohexylcarbodiimide |
| DCM | dichloromethane |
| DMF | N,N-dimethylformamide |
| DMSO | dimethylsulfoxide |
| ES⁺ | electrospray ionisation positive scan |
| ES⁻ | electrospray ionisation negative scan |
| h | hours |
| HOAT | 1-hydroxy-7-azabenzotriazole |
| HOBT | 1-hydroxybenzotriazole |
| HPLC | high pressure liquid chromatography |
| m/z | mass spectrum peak |
| min | minutes |
| MS | mass spectrum |
| NMM | N-methyl morpholine |
| NMR | nuclear magnetic resonance |
| q | quartet |
| s | singlet |
| t | triplet |
| TBTU | 2-(1H-benzotriazol-1-yl)-1,1,3,3- |
| | tetramethyluronium tetrafluoroborate |
| Tf | trifluoromethanesulfonyl |
| TFA | trifluoroacetic acid |
| THF | tetrahydrofuran |
| TLC | thin layer chromatography |
| TS⁺ | thermospray ionisation positive scan |
| WSCDI | 1-(3-dimethylaminopropyl)-3-ethylcarbodiimide |
| | hydrochloride |

The Preparations and Examples that follow illustrate the invention but do not limit the invention in any way. All temperatures are in ° C. Flash column chromatography was carried out using Merck silica gel 60 (9385). Solid Phase Extraction (SPE) chromatography was carried out using Varian Mega Bond Elut (Si) cartridges (Anachem) under 15 mmHg vacuum. Thin layer chromatography (TLC) was carried out on Merck silica gel 60 plates (5729). Melting points were determined using a Gallenkamp MPD350 apparatus and are uncorrected. NMR was carried out using a Varian-Unity Inova 400 MHz nmr spectrometer or a Varian Mercury 400 MHz nmr spectrometer. Mass spectroscopy was carried out using a Finnigan Navigator single quadrupole electrospray mass spectrometer or a Finnigan aQa APCl mass spectrometer.

Conveniently, compounds of the invention are isolated following work-up in the form of the free base, but pharmaceutically acceptable acid addition salts of the compounds of the invention may be prepared using conventional means. Solvates (e.g. hydrates) of a compound of the invention may be formed during the work-up procedure of one of the aforementioned process steps.

Where compounds were prepared in the manner described for an earlier Example, the skilled person will appreciate that it may nevertheless be necessary or desirable to employ different work-up or purification conditions.

### Preparation 1

### tert-Butyl 4-[1H-1,2,3-benzotriazol-1-yl(phenyl)methyl]piperazine-1-carboxylate

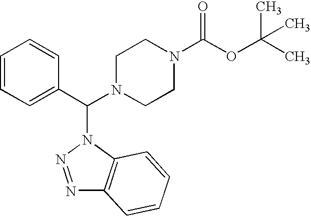

A solution of benzaidehyde (5 ml, 50 mmol), benzotriazole (6 g, 50 mmol) and 1-tert-butyl piperazinecarboxylate (9.3 g, 50 mmol) in toluene (280 ml) was heated at 140° C. for 5 hours, with concommitant removal of water under Dean and Stark conditions. The reaction was then stirred for a further 18 hours at 110° C., and the mixture allowed to cool. The volume of solution was made up to 280 ml with toluene, and the title compound stored as a solution in toluene.

### Preparations 2 to 4

The following preparations of general formula:

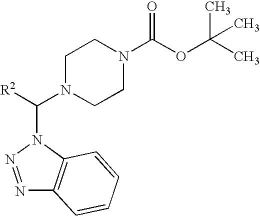

were prepared from benzotriazole, tert-butyl 1-piperazinecarboxylate and the appropriate benzaldehyde, R²COH, following a similar method to that described in preparation 1.

| Prep. No. | R² |
|---|---|
| 2 | 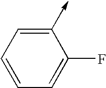 |
| 3 | 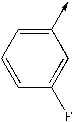 |
| 4 | 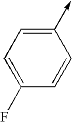 |

### Preparation 5

### tert-Butyl 4-[2-(2-chlorophenyl)-1-phenylethyl]piperazine-1-carboxylate

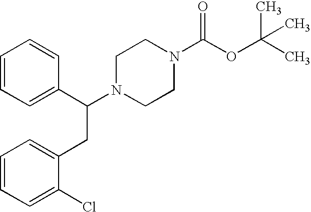

A solution of the compound from preparation 1 (28 ml, 5 mmol) was added dropwise to a solution of 2-chlorobenzylzinc chloride (0.5 M in tetrahydrofuran, 20 ml, 10 mmol) and the reaction stirred at room temperature for 2 hours. The reaction was quenched by the addition of 0.88 ammonia (10 ml) and the mixture partitioned between ethyl acetate and water. The layers were separated, the organic phase washed with 1N sodium hydroxide solution and brine, then dried (MgSO₄) and evaporated under reduced pressure. The crude product was purified by column chromatography on silica gel using an elution gradient of cyclohexane:ethyl acetate (100:0 to 80:20) to provide the title compound as a colourless oil, 1.94 g.

¹H nmr (CDCl₃, 400 MHz) δ: 1.40 (s, 9H), 2.50 (m, 4H), 2.90 (m, 1H), 3.40 (m, 4H), 3.60 (m,1H), 3.68 (m,1H), 6.74 (d,1H), 6.96 (m,1H), 7.03 (m, 1H), 7.12 (m, 2H), 7.18-7.30 (m, 5H). LRMS: m/z (ES⁺) 401 [MH]⁺

### Preparation 6

### tert-Butyl 4-{1-phenyl-2-[2-(trifluoromethoxy)phenyl]ethyl}piperazine-1-carboxylate

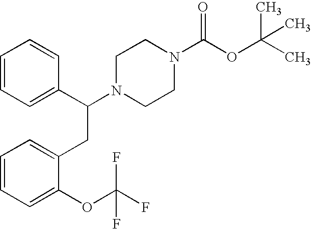

1,2-Dibromoethane (0.05 ml, 0.58 mmol) was added to a suspension of zinc (490 mg, 7.5 mmol) in tetrahydrofuran (15 ml) and the mixture heated at reflux for 2 minutes, then allowed to cool. Chlorotrimethylsilane (0.13 ml, 1 mmol) was added, the mixture sonicated and a solution of 2-(trifluoromethoxy)benzyl bromide (1.28 g, 5 mmol) in tetrahydrofuran (10 ml) added dropwise over 5 minutes. Sonication was continued for a further 30 minutes, and the mixture then stirred for an hour. A solution of the compound from preparation 1 (14 ml, 2.5 mmol) was added and the reaction stirred for 2 hours. The reaction was quenched by the addition of 0.88 ammonia solution (8 ml) and the mixture partitioned between ethyl acetate (20 ml) and water (20 ml). The layers were separated, the organic phase washed with 1M sodium hydroxide solution, dried (MgSO₄) and evaporated under reduced pressure. The crude product was purified by column chromatography on silica gel using an eluton gradient of pentane:ethyl acetate (95:5 to 84:16) to give the title compound as a colourless oil, 859 mg.

¹H nmr (CDCl₃, 400 MHz) δ: 1.42 (s, 9H), 2.43 (m, 4H), 2.88 (dd, 1H), 3.38 (m, 4H), 3.45 (dd, 1H), 3.58 (dd, 1H), 6.86 (d, 1H), 7.00 (m, 1H), 7.10 (m, 4H), 7.20 (m, 3H).

LRMS: m/z (ES⁺) 473 [MNa]⁺

### Preparations 7 to 10

The following compounds of general formula shown below:

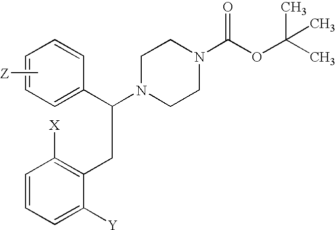

were prepared as described in preparation 6 using the compounds of preparations 1,2 and 4 and the appropriate benzyl bromides.

| Prep. | | | | | |
|---|---|---|---|---|---|
| No | X | Y | Z | Form | Data |
| 7 | F | Cl | H | colour- | ¹H nmr(CDCl₃, 400MHz) δ: |
| | | | | less | 1.40(s, 9H), 2.35(m, 2H), 2.51(m, |
| | | | | oil | 2H), 3.05(m, 1H), 3.38(m, 4H), |
| | | | | | 3.50(m, 1H), 3.79(m, 1H), 6.81(dd, |
| | | | | | 1H), 7.03(m, 2H), 7.21(m, 5H). |
| | | | | | LRMS: m/z (APCI⁺) 419[MH]⁺ |
| 8^{a} | OCHF₂ | H | H | yellow | ¹H nmr(CDCl₃, 400MHz) δ: |
| | | | | oil | 1.42(s, 9H), 2.42(m, 4H), 2.88(m, |
| | | | | | 1H), 3.40(m, 6H), 3.60(m, 1H), |
| | | | | | 6.84-7.00(m, 3H), 7.10-7.39(m, 6H). |
| 9^{a} | OCF₃ | H | 2-F | orange | ¹H nmr(CDCl₃, 400MHz) δ: |
| | | | | gum | 1.42(s, 9H), 2.42(m, 4H), 3.00(m, |
| | | | | | 1H), 3.40(m, 5H), 4.18(m, 1H), |
| | | | | | 6.95(m, 1H), 7.02-7.40(m, 7H). |
| | | | | | LRMS: m/z APCI⁺ 469[MH]⁺ |
| 10^{a} | OCF₃ | H | 4-F | orange | ¹H nmr(CDCl₃, 400MHz) δ: |
| | | | | gum | 1.42(s, 9H), 2.40(m, 4H), 2.82(m, |
| | | | | | 1H), 3.33-58(m, 6H), 6.80(m, 1H), |
| | | | | | 6.90(m, 2H), 6.98-7.38(m, 5H). |
| | | | | | LRMS: m/z 469[MH]⁺ |

^{a}isolated without column chromatography

### Preparation 11

### tert-Butyl 4-{1-(3-fluorophenyl)-2-[2-(trifluoromethoxy)phenyl]ethyl}piperazine-1-carboxylate

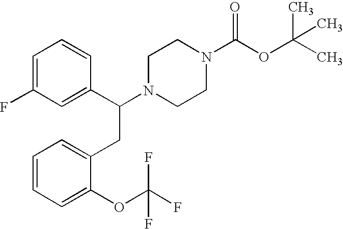

2-(Trifluoromethoxy)benzyl bromide (20.5 g, 80.5 mmol) was added to a cooled (−25° C.) solution of Rieke® zinc (90 ml, suspension of 5.0 g Zn in 100 ml tetrahydrofuran, 68.8 mmol), and the mixture stirred for 1 hour. This solution was added portionwise to a solution of the compound from preparation 3 (220 ml, 0.15M in toluene, 32.2 mmol) and the reaction stirred at room temperature for 18 hours. The reaction was quenched by the addition of 0.88 ammonia (100 ml) and the mixture diluted with water (350 ml) and ethyl acetate (200 ml) and the phases separated. The organic layer was dried (MgSO₄) and evaporated under reduced pressure to give the title compound as a brown oil.

¹H nmr (CDCl₃, 400 MHz) δ: 1.42 (s, 9H), 2.42 (m, 4H), 2.82 (m,, 1H), 3.36-3.50 (m, 5H), 3.58 (m, 1H), 6.82-7.30 (m, 8H).

LRMS: m/z APCl⁺ 469 [MH]⁺

### Preparation 12

### 1-(Chloromethyl)-2-ethoxybenzene

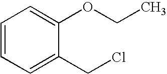

Thionyl chloride (115 ml, 1.48 mol) was added dropwise to a solution of 2-ethoxybenzyl alcohol (214 g, 1.41 mol) in dichloromethane (1.3L) and once addition was complete, the reaction was heated under reflux for 3 hours. The cooled mixture was concentrated under reduced pressure and the residue azeotroped with tetrahydrofuran. The crude product was purified by distillation to give the title compound as a colourless oil, 190 g. (b.p. 80° C. at 2 mmHg).

¹H nmr (CDCl₃, 400 MHz) δ: 1.45 (t,- 3H), 4.10 (q, 2H), 4.70 (s, 2H), 6.85 (d, 1H), 6.90 (dd,1H), 7.25 (dd,1H), 7.35 (d,1H).

### EXAMPLE 1

### 1-{1-Phenyl-2-[2-(trifluoromethoxy)phenyl]ethyl}piperazine ditrifluoroacetate

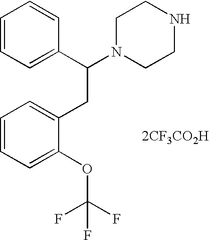

Trifluoroacetic acid (1.9 ml) was added to a solution of the compound from preparation 6 (859 mg, 1.91 mmol) in dichloromethane (10 ml) and the mixture stirred at room temperature for 2 hours. The reaction mixture was concentrated under reduced pressure and the residue azeotroped with toluene. The solid was triturated with ether, to afford the title compound as a white powder, 296 mg.

¹H nmr (CD₃OD, 400 MHz) δ: 2.86 (m, 4H), 3.12 (dd,1H), 3.22 (m, 4H), 3.56 (dd, 1H), 3.95 (dd, 1H), 7.08 (m, 2H), 7.18-7.35 (m, 7H).

LRMS: m/z (ES⁺) 351 [MH]⁺

### EXAMPLES 2 to 3

The following examples of general formula:

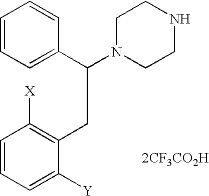

were prepared from the appropriate protected piperidines following the method described in example 1.

| Ex. | | | |
|---|---|---|---|
| No | X | Y | Data |
| 2 | F | Cl | ¹H nmr(CD₃OD, 400MHz)δ: 2.72(m, 2H), |
| | | | 2.90(m, 2H), 3.19(m, 5H), 3.60(dd, 1H), 4.00(dd,1H), |
| | | | 6.98(m, 1H), 7.19(m, 2H), 7.28(m, 5H). |
| | | | LRMS: m/z(APCI⁺)319[MH]⁺ |
| | | | Microanalysis found: C, 48.31; H, 4.06; N, 5.07. |
| | | | C₁₈H₂₀ClFN₂; 2CF₃CO₂H |
| | | | requires C, 48.22; H, 4.03; N, 5.12%. |
| 3^{a} | Cl | H | ¹H nmr(CD₃OD, 400MHz)δ: 2.94(m, 4H), |
| | | | 3.18(dd, 1H), 3.25(m, 4H), 3.63(dd, 1H), 4.02(dd, |
| | | | 1H), 6.93(d, 1H), 7.00(dd, 1H), 7.10(dd, 1H), |
| | | | 7.26(m, 6H). |
| | | | LRMS: m/z(APCI⁺)301[MH]⁺ |
| | | | Microanalysis found: C, 49.72; H, 4.32; N, 5.24. |
| | | | C₁₈H₂₁ClN₂; 2CF₃CO₂H |
| | | | requires C, 49.96; H, 4.38; N, 5.30%. |

^{a}1:1 ratio by volume of trifluoroacetic and dichloromethane used.

### EXAMPLE 4

### 1-{1-(3-Fluorophenyl)-2-[2-(trifluoromethoxy)phenyl]ethyl}piperazine

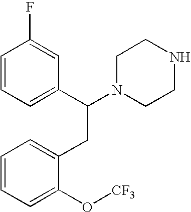

Trifluoroacetic acid (16.4 ml, 213.5 mmol) was added dropwise to an ice-cooled solution of the compound from preparation 11(10.0 g, 21.3 mmol) in dichloromethane (110 ml) and the solution stirred at room temperature for 18 hours. The reaction mixture was concentrated under reduced pressure and the residue azeotroped with toluene and dichloromethane. The product was partitioned between ether (300 ml) and sodium hydroxide solution (500 ml, 2M) and the layers separated. The organic phase was dried (MgSO₄) and evaporated under reduced pressure. The crude product was purified by column chromatography on silica gel using an elution gradient of ethyl acetate:pentane:dichloromethane:methanol:0.88 ammonia (20:80:0:0:0 to 0:0:90:10:1) to give the title compound as a brown oil, 3.5 g.

¹H nmr (CDCl₃, 400 MHz) δ: 2.79 (m, 5H), 3.18 (m, 4H), 3.40 (dd, 1H), 3.62 (dd, 1H), 6.82 (m, 3H), 6.90 (m, 1H), 7.02 (m, 1H), 7.19 (m, 3H).

LRMS: m/z APCI⁺ 369 [MH]⁺

### EXAMPLE 5

### 1-{2-[2-(Difluoromethoxy)phenyl]-1-phenylethyl}piperazine

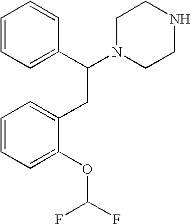

Trifluoroacetic acid (47 ml) was added dropwise to an ice-cooled solution of the compound from preparation 8 (26.4 g, 61 mmol) in dichloromethane (50 ml) and the reaction stirred at room temperature for 3 hours. The mixture was concentrated under reduced pressure and the residue azaeotroped with toluene and dichloromethane. The product was triturated with ether and the resulting solid filtered off and dried. The solid was partitioned between dichloromethane (200 ml) and saturated sodium bicarbonate solution (100 ml), then sodium hydroxide (6M) added until complete dissolution occurred, and the layers separated. The aqueous phase was extracted with dichloromethane (100 ml) and the combined organic solutions dried (Na₂SO₄) and evaporated under reduced pressure. The residual gum was dissolved in dichloromethane, the solution cooled in ice, and ethereal hydrochloric acid added. The solution was evaporated under reduced pressure, the residue azeotroped with dichloromethane and the product recrystallised from ethanol. This product was partitioned between sodium hydroxide solution (6M) and dichloromethane, the layers separated and the organic phase dried (Na₂SO₄) and evaporated under reduced pressure to afford the title compound as an oil, 12 g.

¹H nmr (CDCl₃, 400 MHz) δ: 2.45 (m, 4H), 2.84 (m, 5H), 3.45 (m, 2H), 6.23 (t, 1H), 6.83 (m, 3H), 7.10 (m, 3H), 7.22 (m, 3H).

LRMS: m/z APCl⁺ 333 [MH]⁺

### EXAMPLE 6

### 1-{1-(4-Fluorophenyl)-2-[2-(trifluoromethoxy)phenyl]ethyl}piperazine

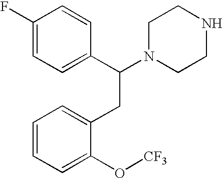

The title compound was obtained as a gum in 21% yield from the compound from preparation 10, following a similar procedure to that described in example 5, except the compound was additionally purified by column chromatography on silica gel using dichloromethane:methanol:0.88 ammonia (90:10:1).

¹H nmr (CDCl₃, 400 MHz) δ: 2.16 (br s, 1H), 2.45 (m, 4H), 2.80 (m, 1H), 2.90 (m, 4H), 3.47 (m, 2H), 6.85 (m, 3H), 7.03 (m, 3H), 7.15 (m, 2H).

LRMS : m/z APCl⁺ 369 [MH]⁺

### EXAMPLE 7

### 1-{1-(2-Fluorophenyl)-2-[2-(trifluoromethoxva)henyl]ethyl}piperazine

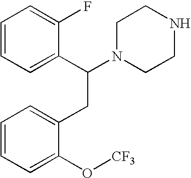

The title compound was obtained from the title compound of preparation 9 following a similar procedure to that described in example 6.

¹H nmr (CDCl₃, 400 MHz) δ: 2.12 (br s,1H), 2.50 (m, 4H), 2.84 (m, 1H), 2.98 (m, 4H), 3.42 (m, 2H), 4.10 (m, 1H), 6.90 (dd, 1H), 7.10 (m, 6H), 7.53 (m,1H).

LRMS: m/z APCl⁺ 369 [MH]⁺

### EXAMPLE 8

### (−)-1-{2-[2-(Difluoromethoxy)phenyl]-1-phenylethyl}piperazine dihydrochloride

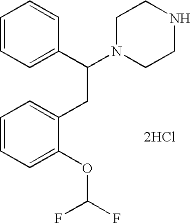

A portion of the compound from example 5 was further purified by HPLC using a Chiralcel OD 250 column and isopropyl alcohol:hexane:diethylamine (10:90:0.2) to provide enantiomer 1. Further elution provided the second enantiomer. The product was purified by column chromatography on silica gel using dichloromethane:methanol:0.88 ammonia (95:5:0.5) as eluant to give the free base of the title compound. This gum was dissolved in dichloromethane, the solution cooled in ice and treated with ethereal hydrochloric acid. The solution was evaporated under reduced pressure to afford the title compound as a white solid.

¹H nmr (CD₃OD, 400 MHz) δ: 3.40-3.58 (m, 4H), 3.62 (m, 4H), 3.82 (dd, 1H), 4.63 (m, 1H), 6.68-7.01 (m, 3H), 7.06 (m, 1H), 7.19 (dd, 1H), 7.40 (m, 3H), 7.52 (d, 2H).

LRMS: m/z APCl⁺ 333 [MH]⁺

[α]_{D}=−58.67 (c=0.1, methanol)

Microanalysis found: C, 54.17; H, 5.87; N, 6.53. C₁₉H₂₂F₂N₂O;2HCl:H₂O requires C, 53.91; H, 6.19; N, 6.82%.

### EXAMPLES 9 and 10

### (−) and (+)-1-[2-(2-Chlorophenyl)-1-phenylethyl]piperazine dihydrochloride

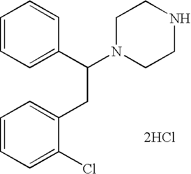

The compound from example 3 (1.2 g) was dissolved in methanol and the solution treated with 1 M sodium hydroxide solution (20 ml), the solution stirred at room temperature for 30 minutes then concentrated under reduced pressure. The aqueous solution was extracted with ethyl acetate (×2), the combined organic extracts washed with 1N sodium hydroxide solution, brine, then dried (Na₂SO₄) and evaporated under reduced pressure. The residual yellow oil was further purified by HPLC using a chiralcel OJ 250 column and hexane:ethanol:diethylamine (80:20:0.2) as eluant to provide the free base of example 9. This was purified by column chromatography on silica gel using dichloromethane:methanol:0.88 ammonia (100:0:0 to 90:10:1) to give a colourless oil. This was dissolved in dichloromethane (4 ml) and the solution treated with ethereal hydrochloric acid (10 ml, 1M), the solution stirred for 30 minutes, then evaporated under reduced pressure to give the title compound of example 9 as a white solid, 320 mg.

¹H nmr (CD₃OD, 400 MHz) δ: 3.32 (m, 2H), 3.48 (m, 2H), 3.66 (m, 5H), 3.95 (dd, 1H), 4.75 (dd, 1H), 6.94 (d, 1H), 7.00 (dd, 1H), 7.15 (dd, 1H), 7.30 (d,1H), 7.41 (m, 3H), 7.57 (m, 2H).

LRMS: m/z (ES⁺) 301 [MH]⁺

Microanalysis found: C, 56.01; H, 6.16; N, 7.09.

C₁₈H₂₁ClN₂;2HCl;0.15CH₂Cl₂ requires C, 56.40; H, 6.08; N, 7.25%.

[α]_{D}=−88.22 (c=0.2, methanol)

Further elution of the chiralcel OJ column provided the free base of example 10. This was treated as described for example 9 to provide the title compound of example 10.

¹H nmr (CD₃OD, 400 MHz) δ: 3.34 (m, 2H), 3.45 (m, 2H), 3.62 (m, 5H), 3.95 (dd, 1H), 4.75 (dd, 1H), 6.94 (d, 1H), 7.00 (dd, 1H), 7.15 (dd, 1H), 7.30 (d,1H), 7.40 (m, 3H), 7.57 (m, 2H).

LRMS: m/z (APCl⁺) 301 [MH]⁺

Microanalysis found: C, 56.79; H, 6.21; N, 7.17.

C₁₈H₂₁ClN₂;2HCl;0.10CH₂Cl₂ requires C, 56.87; H, 6.12; N, 7.33%. [α]_{D}=+87.32 (c=0.2, methanol)

### EXAMPLE 11

### 1-[2-(2-Methoxyphenyl)-1-phenylethyl]piperazine dihydrochloride

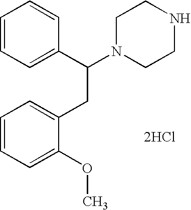

A crystal of iodine was added to a suspension of magnesium turnings (2.43 g, 100 mmol) in tetrahydrofuran (120 ml) and the mixture heated at reflux for 10 minutes. The mixture was diluted with additional tetrahydrofuran (80 ml) and a solution of 2-methoxybenzyl chloride (11.73 g, 75 mmol) in tetrahydrofuran (20 ml) was added dropwise via a pressure equalising dropping funnel over 1 hour, so as to maintain the reaction at reflux. The reaction was heated under reflux for a further hour, then allowed to cool to room temperature.

This solution was cooled to −70° C., and a solution of the compound from preparation 1 (140 ml, 0.25M in toluene, 35 mmol) added dropwise over 20 minutes. The reaction was stirred for a further 15 minutes, then warmed to 0° C. over 30 minutes and poured onto a mixture of ice (300 g) and concentrated hydrochloric acid (100 ml). This mixture was stirred for 2 hours, additional concentrated hydrochloric acid (200 ml) added and the mixture stirred for a further hour. The mixture was filtered, washing through with ether (2×250 ml) and the filtrate separated. The aqueous layer was basified carefully using 0.88 ammonia and this solution extracted with dichloromethane (4×250 ml) and the combined organic extracts dried (MgSO₄) and evaporated under reduced pressure. The product was dissolved in ethyl acetate (300 ml) and washed with 20% aqueous potassium carbonate solution (3×200 ml), then dried (MgSO₄) and evaporated under reduced pressure. The residual yellow oil was purified by column chromatography on silica gel using an elution gradient of dichloromethane:methanol:0.88 ammonia (95:5:0.5 to 90:10:1) to give a pale orange oil, in addition to a yellow oil (product with minor impurity). A sample of this product was converted to the dihydrochloride salt to provide the title compound of example 11.

¹H nmr (D₂O, 400 MHz) δ: 3.22-3.68 (m, 1 OH), 4.60 (m, 1H), 6.58 (m, 1H), 6.84 (m, 2H), 7.14 (dd, 1H), 7.36 (m, 5H).

LRMS: m/z (ES⁺) 297 [M+2H]⁺

### EXAMPLE 12

### (−)-1-[2-(2-Methoxyphenyl)-1-phenylethyl]piperazine dihydrochloride

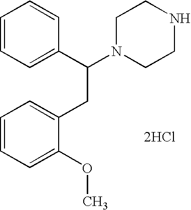

The free base of example 11 was dissolved in dichloromethane, treated with trifluoroacetic acid and the solution evaporated under reduced pressure. The solid was triturated with water and the resulting crystals dried and recrystallised from hot ethyl acetate. These white-crystals were partitioned between ethyl acetate (70 ml) and 1N sodium hydroxide solution (150 ml), the organic phase washed with 1N sodium hydroxide solution (20 ml), then dried (MgSO₄) and evaporated under reduced pressure. The product was purified by HPLC using a Chiralcel OD 250 (20 mm) column and hexane:isopropyl alcohol:diethylamine (80:20:0.3) as eluant, to provide enantiomer 1. Further elution provided enantiomer 2, which was repurified by column chromatography on silica gel using dichloromethane:methanol:0.88 ammonia (90:10:1). The resulting gum was dissolved in methanol (4 ml) the solution treated with 1N hydrochloric acid (2 ml) and then evaporated under reduced pressure to provide the title compound of example 12,

¹H nmr (D₂O, 400 MHz) δ: 3.19 (m, 1H), 3.32 (m, 2H), 3.41 (m, 4H), 3.56 (dd,1H), 4.57 (m, 1H), 6.58 (dd,1H), 6.78 (m, 2H), 7.01 (dd,1H), 7.24 (m, 5H).

LRMS: m/z (APCl⁺) 297 [MH]⁺

Microanalysis found: C, 61.07; H, 7.11; N, 7.44. C₁₈H₂₁ClN₂;2HCl;0.2H₂O requires C, 61.19; H, 7.14; N, 7.51%.

[α]_{D}=−105.0 (c=0.112, methanol)

### EXAMPLES 13 and 14

### (+) and (−)1-[2-(2-Ethoxyphenyl)-1-phenylethyl]piperazine dihydrochloride

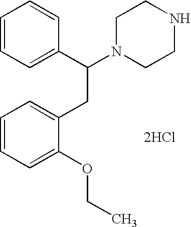

A crystal of iodine was added to a suspension of magnesium turnings (13.5 g, 0.56 mol) in tetrahydrofuran (200 ml) and the mixture heated at reflux until decolourisation occurred. The mixture was diluted with additional tetrahydrofuran (200 ml) and a solution of the benzyl chloride from preparation 12 (85.25 g, 0.5 mol) in tetrahydrofuran (400 ml) was added dropwise via a dropping funnel over 2 hours, so as to maintain the reaction at reflux. The reaction was heated under reflux for a further 2 hours, then allowed to cool to room temperature.

This solution was cooled to −78° C., and a solution of the compound from preparation 1 (98.37 g, 0.25 mol) (prepared by evaporation under reduced pressure of the solution from preparation 1) in tetrahydrofuran (800 ml) added dropwise over 35 minutes, so as to maintain the temperature below −65° C. The reaction was stirred for a further 30 minutes, then warmed to 0° C. over 1 hour and poured slowly onto a mixture of ice (500 g), concentrated hydrochloric acid (100 ml) and toluene (1.5L), so that the temperature was maintained below 15° C. Additional concentrated hydrochloric acid (650 ml) was added portionwise with cooling and once addition was complete, the mixture was stirred at room temperature for 42 hours. The mixture was separated and the aqueous layer was washed with toluene (2×750 ml), cooled in an ice-bath then basified carefully using 0.88 ammonia (520 ml). This solution was extracted with dichloromethane (3×1 L, 3×750 ml) and the combined organic extracts washed with water (1 L) and evaporated under reduced pressure. The residual brown oil was dissolved in ethyl acetate (1.2 L), and the solution washed with 20% aqueous potassium carbonate solution (4×500 ml), dried (MgSO₄) and evaporated under reduced pressure. The product was dissolved in ethanol (700 ml), 1M ethereal hydrochloric acid (700 ml) added and the solution evaporated under reduced pressure to give an orange solid. This was recrystallised from hot ethanol to provide the racemate of the title compounds as a white solid, obtained in three crops, 105.3 g in total. A portion of this compound was further purified by HPLC using a chiralcel OD column, and hexane:isopropyl alcohol:diethylamine (70:30:0.3) as eluant to give the free base of example 13. This was further purified by column chromatography on silica gel using dichloromethane:methanol:0.88 ammonia (90:10:1) as eluant, the product treated with ethereal hydrochloric acid and dried at 70° C. to provide the title compound of example 13.

¹H nmr (D₂O, 400 MHz) δ: 1.36 (t, 3H), 3.26-3.45 (m, 4H), 3.51 (m, 4H), 3.67 (m, 2H), 3.95 (m,1H), 4.05 (m, 1H), 4.60 (m,1H), 6.65 (m,1H), 6.84 (m, 2H), 7.08 (m,1H), 7.32 (m, 5H).

LRMS: m/z (APCl⁺) 311 [MH]⁺

Microanalysis found: C, 62.01; H, 7.48; N, 7.19. C₂₀H₂₆N₂O;2HCl;0.25H₂O requires C, 61.93; H, 7.41; N, 7.22%.

[α]_{D}=+84.22 (c=0.2, methanol)

Further elution of the chiralcel OD column provided the free base of example 14. This was further purified by column chromatography on silica gel using dichloromethane: methanol:0.88 ammonia (90:1 0:1) as eluant, the product treated with 1M ethereal hydrochloric acid and dried at 70° C. to provide the title compound of example 14.

¹H nmr (D₂O, 400 MHz) δ: 1.37 (t, 3H), 3.24-3.38 (m, 4H), 3.50 (m, 4H), 3.61 (m, 2H), 3.96 (m,1H), 4.07 (m,1H), 4.55 (m,1H), 6.66 (m,1H), 6.85 (m, 2H), 7.08 (m, 1H), 7.32 (m, 5H).

LRMS: m/z (APCl⁺) 311 [MH]⁺

[α]_{D}=−97.02 (c=0.2, methanol)

Microanalysis found: C, 61.22; H, 7.47; N, 7.11. C₂₀H₂₆N₂O;2HCl;0.5H₂O requires C, 61.22; H, 7.45; N, 7.14%.

A portion of this compound, (257 mg, 0.65 mmol) was dissolved in sodium hydroxide solution (20 ml, 1M) and the solution extracted with dichloromethane (3×5 ml). The combined organic extracts were dried (MgSO₄) and evaporated under reduced pressure to give a gum. This was dissolved in methanol (20 ml), and a solution of succinic acid (77 mg, 0.65 mmol) in methanol (5 ml) added. The solution was stirred until homogeneous and then evaporated under reduced pressure. The residue was triturated with ethyl acetate (10 ml) and the resulting solid was dried to give a white solid. This solid was recrystallised twice from acetone to afford the succinate salt of example 14.

¹H nmr (DMSO-d₆, 400 MHz) δ: 1.38 (t, 3H), 2.23 (m, 4H), 2.48 (m, 4H), 2.85 (m, 4H), 3.22 (m, 2H), 3.78 (m,1H), 3.88-4.02 (m, 2H), 6.64 (dd,1H), 6.81 (d, 1H), 6.85 (d, 1H), 7.02 (dd, 1H), 7.18 (m, 3H), 7.24 (m, 2H).

### EXAMPLE 15

The NRl Ki and SRl Ki values of the compounds of Examples 1, 2, 5, 8 and 14 were determined as follows. The results are set out below in Table 1.

Biological Activity

The compounds were tested for biological activity by their ability to compete with and inhibit the binding of [³H]Nisoxetine to the human noradrenaline transporter, [³H]Citalopram to the human serotonin transporter and [³H]WIN-35428 to the human dopamine transporter as follows.

(i) Membrane Preparation

Human embryonic kidney cells (HEK-293) stably transfected with either the human serotonin transporter (hSERT), noradrenaline transporter (hNET) or dopamine transporter (hDAT) were cultured under standard cell culture techniques (cells were grown at 37° C. and 5% CO₂ in either Dulbecco's Modified Eagle's Medium (DMEM) culture media supplemented with 10% dialysed foetal calf serum (FCS), 2 mM L-glutamine and 250 μg/ml geneticin (hSERT and hNET cells) or DMEM-culture media supplemented with 5% FCS, 5% new-born calf serum, 2 mM L-glutamine and 2.5 mg/ml puromycin (hDAT cells)). Cells were harvested, pelleted by centrifugation and re-suspended in ice-cold membrane prep buffer. The cell suspension was then homogenized, large particulate matter removed by low speed centrifugation and the supernatant re-centrifuged (35,000× g, 30 minutes at 4° C.). The pelleted membranes were re-suspended in membrane prep buffer, protein concentrations measured (Sigma protein kit) and the membrane suspension stored frozen in aliquots.

(i) Determination of Inhibitor Potency

Prior to assay, membranes containing the respective human transporter protein were pre-coupled to the appropriate scintillation-proximity assay (SPA) bead, i.e., PVT WGA SPA beads (Amersham) for hNET and hDAT and YSi WGA SPA beads (Amersham) for hSERT, so as to minimise ligand depletion and maximise the assay window for the corresponding [³H] ligand. SPA beads re-suspended (˜50 mg/ml) in assay buffer (1.5×) were pre-coupled with membranes (typically 5-40 μg membrane per mg of bead) by incubating with gentle shaking for 2 hours at 4° C. After coupling, the beads/membranes were collected by centrifugation and washed and re-suspended in assay buffer (1.5×) with gentle stirring at the required concentration for the assay (typically 5-40 mg beads/ml). Also prior to assay, each [³H] ligand was diluted in assay buffer (1 .5×) to give a stock concentration of 3× the final assay concentration (typical final concentrations=12 nM [³H]Nisoxetine (Amersham), 2.5 nM [³H]Citalopram (Amersham) and 10 nM [³H]WIN-35428 (Perkin Elmer), which were confirmed by scintillation counting). Finally, all test compounds were dissolved in 100% DMSO at 4 mM and diluted down in 1% DMSO in water to give appropriate test concentrations.

Assays were carried out in 384-well NBS plates (Costar). For each assay, 20 μl of the appropriate dilution of either test compound, a standard inhibitor (positive control) or compound vehicle (DMSO in water; final DMSO concentration was 0.25% in each assay well) was added to 20 μl of the appropriate stock of [³H] ligand. 20 μl of the corresponding bead/membrane preparation was then added and the plate sealed prior to incubation with shaking for 1 hour. The assay plates were then incubated at room temperature for at least a further 6 hours (to attain equilibrium) with dark adaptation, before direct scintillation counting.

Potency of test compounds was quantified as IC₅₀ values (concentration of test compound required to inhibit the specific binding of radio-labelled ligand to the respective transporter protein by 50% relative to maximum (compound vehicle only) and minimum (complete inhibition by standard inhibitor) responses). The Ki value was derived for each compound by conversion of the IC₅₀ value using the Cheng-Prusoff equation and the experimentally measured free ligand concentration and Kd for the batch of membrane used in assay (typical Kd values: ˜30 nM Nisoxetine, ˜8 nM Citalopram and ˜15 nM WIN-35428).

(iii) Membrane Prep Buffer

HEPES (20 mM) HEPES

1 complete protease inhibitor tablet (Roche)/50 ml pH 7.4 at room temperature, store at 4° C.

Assay Buffer (1.5× assay concentration)

HEPES (30 mM)

NaCl (180 mM)

pH 7.4 at room temperature, store at 4° C.

(iv) Summary of Assay Parameters

| | hNET assay | hSERT assay | hDAT assay |
|---|---|---|---|
| Transporter | hNET/PVT | hSERT/YSi | hDAT/PVT |
| membrane/SPA | WGA | WGA | WGA |
| bead type | | | |
| Ligand/ | ³H-Nisoxetine | ³H-citalopram | ³H- WIN-35428 |
| concentration | (12 nM) | (2.5 nM) | (10 nM) |
| Incubation | 7 | 7 | 7 |
| time (hrs) | | | |

TABLE 1

| Compound | SRI Ki (nM) | NRI Ki (nM) |
|---|---|---|
| Example 1 | 27 | 62 |
| Example 2 | 16 | 35 |
| Example 5 | 8 | 28 |
| Example 8 | 6 | 14 |
| Example 14 | 9 | 19 |

1. A compound according to Formula I: 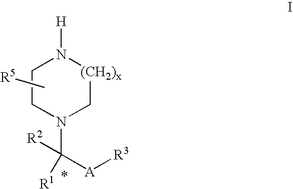
and pharmaceutically and/or veterinarily acceptable derivatives thereof, wherein:
R¹ is H;
R² is aryl, het, C₃₋₈cycloalkyl, C₁₋₆alkyl, (CH₂)₂aryl or R⁴, wherein each of the cycloalkyl, aryl, het and R⁴ groups is optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkyl-SO₂-, C₁₋₄alkyl-S-C₁₋₄alkyl, C₁₋₄alkyl-S—, C₁₋₄alkylNR¹⁰R¹¹ and NR¹⁰R¹¹;
or R¹ and R², together with the carbon atom to which they are bound, form a 5- or 6-membered carbocyclic ring or a 5- or 6-membered heterocyclic ring containing at least one N, O or S heteroatom;
where R¹ and R² are different, * represents a chiral centre;
R³ is aryl, het or R⁴, each optionally substituted by at least one substituent independently selected from C₁₋₆alkyl, C₁₋₆alkoxy, het, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂, C₁₋₄alkyl-S-C₁₋₄alkyl, C₁₋₄alkyl-S-, C₁₋₄alkylNR¹⁰R¹¹ and NR¹⁰R¹¹;
R⁴ is a phenyl group fused to a 5- or 6-membered carbocyclic group, or a phenyl group fused to a 5- or 6-membered heterocyclic group containing at least one N, O or S heteroatom;
R⁵ is H or C₁₋₆alkyl;
R¹⁰ and R¹¹ are the same or different and are independently H or C₁₋₄alkyl;
A is an unsubstituted methylene group;
x is an integer from 1 to 3;
y is 1 or 2;
z is an integer from 1 to 3;
aryl is phenyl, naphthyl, anthracyl or phenanthryl; and
het is an aromatic or non-aromatic 4-, 5- or 6-membered heterocycle which contains at least one N, O or S heteroatom, optionally fused to a 5- or 6-membered carbocyclic group or a second 4-, 5- or 6-membered heterocycle which contains at least one N, O or S heteroatom, provided that when R¹ is H, R² is phenyl, A is CH₂ and x is 1, R³ is not 3-hydroxyphenyl or 3-(C₁₋₄alkoxy)phenyl, or a pharmaceutically acceptable salt thereof.

2. (canceled)

3. A compound or a pharmaceutically acceptable salt thereof according to claim 1, wherein R² is aryl, het or C₃₋₈cycloalkyl, each optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂ and C₁₋₄alkyl-S-C₁₋₄alkyl.

4. A compound or a pharmaceutically acceptable salt thereof according to claim 3, wherein R² is aryl optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C_{1- 4}alkoxy-C₁₋₆alkyl, C₁₋₄alkoxy-C₁₋₄alkoxy, SCF₃, C₁₋₆alkylSO₂ and C₁₋₄alkyl-S-C₁₋₄alkyl.

5. A compound or a pharmaceutically acceptable salt thereof according to claim 4, wherein R² is phenyl optionally substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂ and C₁₋₄alkyl-S-C₁₋₄alkyl.

6. A compound or a pharmaceutically acceptable salt thereof according to claim 1, wherein R³ is aryl or R⁴, each substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂ and C₁₋₄alkyl-S-C₁₋₄alkyl.

7. A compound or a pharmaceutically acceptable salt thereof according to claim 6, wherein R³ is phenyl substituted by at least one substituent independently selected from C₁₋₆alkyl, C₁₋₆alkoxy, OH, halo, CF₃, OCF₃, OCHF₂, O(CH₂)_{y}CF₃, CN, CONH₂, CON(H)C₁₋₆alkyl, CON(C₁₋₆alkyl)₂, hydroxy-C₁₋₆alkyl, C₁₋₄alkoxy-C₁₋₆alkyl, C₁₋₄alkoxy-C₁₋₄alkoxy, SCF₃, C₁₋₆alkylSO₂ and C₁₋₄alkyl-S-C₁₋₄alkyl.

8. A compound or a pharmaceutically acceptable salt thereof according to claim 1, wherein R⁵ is H or C₁₋₆alkyl.

9. A compound or a pharmaceutically acceptable salt thereof according to claim 1, wherein x is 1.

10. A compound or a pharmaceutically acceptable salt thereof according to claim 1 which is (+) or (−)-1-[2-(2-Ethoxyphenyl)-1-phenylethyl]piperazine.

11. A compound or a pharmaceutically acceptable salt thereof according to claim 1 which is selected from the group consisting of:
1-{1-Phenyl-2-[2-(trifluoromethoxy)phenyl]ethyl}piperazine;
1-{1-Phenyl-2-[2-chloro-6-fluorophenyl]ethyl}piperazine;
1-{1-Phenyl-2-[2-chlorophenyl]ethyl}piperazine;
1-{1-(3-Fluorophenyl)-2-[2-(trifluoromethoxy)phenyl]ethyl}piperazine;
1-{2-[2-(Difluoromethoxy)phenyl]-1-phenylethyl}piperazine;
1-{1-(4-Fluorophenyl)-2-[2-(trifluoromethoxy)phenyl]ethyl}piperazine;
1-{1-(2-Fluorophenyl)-2-[2-(trifluoromethoxy)phenyl]ethyl}piperazine; and
1-[2-(2-Methoxyphenyl)-1-phenylethyl]piperazine.

12. A pharmaceutical composition comprising a compound or a pharmaceutically acceptable salt thereof as claimed in claims 1 and a pharmaceutically acceptable adjuvant, diluent or carrier.

13. (canceled)

14. (canceled)

15. (canceled)

16. (canceled)

17. (canceled)

18. A method of treatment of a disorder in which the regulation of serotonin or noradrenaline is implicated which comprises administering a therapeutically effective amount of a compound or a pharmaceutically acceptable salt thereof according to claims 1 to a patient in need of such treatment.

19. A method according to claim 18, wherein the regulation of serotonin and noradrenaline is implicated.

20. A method of treatment of urinary disorders, depression, pain, premature ejaculation, ADHD or fibromyalgia, which comprises administering a therapeutically effective amount of a compound or a pharmaceutically acceptable salt thereof according to of claims 1 to a patient in need of such treatment.

21. A method according to claim 20, wherein the urinary disorder is urinary incontinence.

22. A method according to claim 21, wherein the urinary disorder is genuine stress incontinence or stress urinary incontinence.

23. A process for preparing a compound or a pharmaceutically acceptable salt thereof according to claims 1 comprising reacting a compound of Formula III 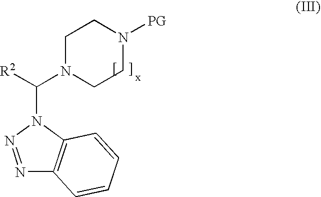
wherein R2 and x are as defined in claims 1 and PG is a protecting group;
with a compound of Formula IV 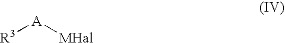
wherein R3 and A are as defined in claims 1, M is a metal selected from Zn and Mg and Hal is a halogen atom selected from chlorine, bromine and iodine;
and deprotecting the resultant compound.

24. (canceled)
